(19) United States
(12) Patent Application Publication
(10) Pub. No.: US 2010/0240657 A1
(43) Pub. Date: Sep. 23, 2010

(54) CHEMICAL COMPOUNDS

(75) Inventors: Ioannis Sapountzis, Vienna (AT); Peter Ettmayer, Vienna (AT); Christian Klein, Vienna (AT); Andreas Mantoulidis, Vienna (AT); Martin Steegmaier, Reutlingen (DE); Steffen Steurer, Vienna (AT); Irene Waizenegger, Vienna (AT)

Correspondence Address: MICHAEL P. MORRIS;BOEHRINGER INGELHEIM USA CORPORATION, 900 RIDGEBURY RD, P. O. BOX 368, RIDGEFIELD, CT, 06877-0368, US

(73) Assignee: BOEHRINGER INGELHEIM INTERNATIONAL GMBH, Ingelheim am Rhein (DE)

(21) Appl. No.: 12/665,776

(22) PCT Filed: Jul. 1, 2008
(86) PCT No.: PCT/EP08/58432

§ 371 Date: Mar. 30, 2010

(30) Foreign Application Priority Data
Jul. 2, 2007 (EP) 07111568.7

(51) Int. Cl. A61K 31/5377; C07C 63/64; A61K 31/192; C07D 401/04; A61K 31/4439; C07D 277/22; A61K 31/427; C07D 263/32; A61K 31/422; C07D 249/06; A61K 31/4196; C07D 403/04; A61K 31/506; C07D 471/04; A61K 31/437; C07C 307/02; A61K 31/18; C07D 413/14; A61K 31/497; C07C 69/78; A61K 31/235; A61P 35/00; A61P 29/00; A61P 37/00

(52) U.S. Cl. 514/236.2; 562/495; 514/568; 546/268.4; 514/340; 548/204; 514/365; 548/236; 514/374; 548/255; 514/383; 544/333; 514/256; 546/121; 514/300; 564/99; 514/605; 544/364; 514/253.1; 544/131; 560/51; 514/544

(57) ABSTRACT

The present invention encompasses compounds of general formula (1) wherein the groups R² to R⁴, L, Q and n are defined as in claim 1, which are suitable for the treatment of diseases characterised by excessive or abnormal cell proliferation, and their use for preparing a pharmaceutical composition with the above-mentioned properties.

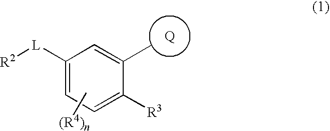

The present invention relates to new compounds of general formula (1)

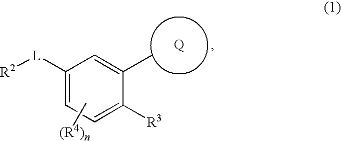

wherein the groups R² to R⁴, L, Q and n have the meanings given in the claims and specification, as well as the tautomers, racemates, enantiomers, diastereomers, mixtures, polymorphs and salts of all these forms and their use as medicaments.

### BACKGROUND TO THE INVENTION

Phenyl-substituted, nitrogen-containing five-ring heteroaryls for inhibiting cytokine production and hence for treating inflammatory diseases are described in WO 2004/050642, WO 2005/056535, WO 2005/090333, WO 2005/115991 and US 2006/0100204.

The aim of the present invention is to indicate new active substances which can be used for the prevention and/or treatment of diseases characterised by excessive or abnormal cell proliferation.

### DETAILED DESCRIPTION OF THE INVENTION

Surprisingly, it has been found that compounds of general formula (1), wherein the groups R² to R⁴, L, Q and n have the meanings given hereinafter, act as inhibitors of specific signal enzymes which are involved in controlling the proliferation of cells. Thus, the compounds according to the invention may be used for example for the treatment of diseases associated with the activity of these signal enzymes and characterised by excessive or abnormal cell proliferation.

The present invention therefore relates to compounds of general formula (1)

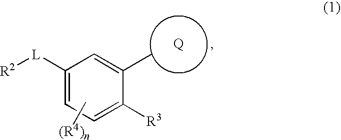

wherein

Q has a partial structure selected from the partial structures (i)-(iv)

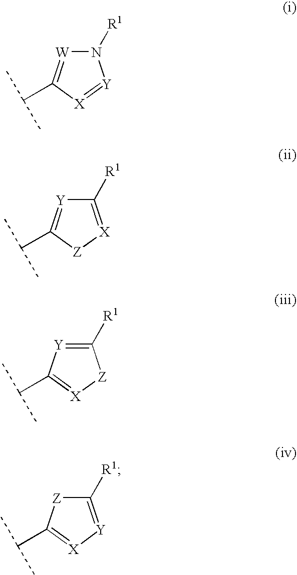

W, X and Y are each independently of one another selected from among ═CR^{5a}— and ═N—;

Z is independently selected in each case from among —NR⁶—, —O— and —S—;

L is selected from among —C(O)NH—, —NHC(O)—, —S(O)NH—, —S(O)₂NH—, —C(NH)NH—, —NHC(NH)—, —NHS(O)— and —NHS(O)₂—;

R¹ denotes a group optionally substituted by one or more identical or different R^{b} and/or R^{c}, selected from among C₃₋₁₀cycloalkyl, C₄₋₁₆cycloalkylalkyl, C₇₋₁₆arylalkyl, 5-12 membered heteroaryl, 6-18 membered heteroarylalkyl and 3-14 membered heterocycloalkyl;

R² is selected from among C₆₋₁₀aryl and 5-12 membered heteroaryl, the above-mentioned groups substituted by one or more identical or different R^{5b}, wherein at least one R^{5b} must be different from hydrogen;

R³ and each R⁴ are each independently selected from among hydrogen, halogen, —CN, —NO₂, —NR^{h}R^{h}, —OR^{h}, —C(O)R^{h}, —C(O)NR^{h}R^{h}, —SR^{h}, —S(O)R^{h}, —S(O)₂R^{h}, C₁₋₄alkyl, C₁₋₄haloalkyl, C₃₋₇cycloalkyl and 3-7 membered heterocycloalkyl;

each R^{5a} and R^{5b} is independently selected from among R^{a} and R^{b};

R⁶ is defined in the same way as R^{a};

n has the value 0, 1, 2 or 3;

each R^{a} independently denotes hydrogen or a group optionally substituted by one or more identical or different R^{b} and/or R^{c}, selected from among C₁₋₆alkyl, 2-6 membered heteroalkyl, C₁₋₆haloalkyl, C₃₋₁₀cycloalkyl, C₄₋₁₆cycloalkylalkyl, C₆₋₁₀aryl, C₇₋₁₆arylalkyl, 5-12 membered hetero-aryl, 6-18 membered heteroarylalkyl, 3-14 membered heterocycloalkyl and 4-14 membered heterocycloalkylalkyl;

each R^{b} denotes a suitable substituent and is independently selected from among —OR^{c}, —SR^{c}, —NR^{c}R^{c}, —ONR^{c}R^{c}, —N(OR^{c})R^{c}, —NR^{g}NR^{c}R^{c}, halogen, —CN, —NC, —OCN, —SCN, —NO, —NO₂, —N₃, —C(O)R^{c}, —C(O)OR^{c}, —C(O)NR^{c}R^{c}, —C(O)SR^{c}, —C(O)NR^{g}NR^{c}R^{c}, —C(O)NR^{g}OR^{c}, —[C(O)]₂NR^{c}R^{c}, —[C(O)NR^{g}]₂R^{c}—C(S)R^{c}, —C(S)OR^{c}, —C(S)NR^{c}R^{c}, —C(S)SR^{c}, —C(NR^{g})R^{c}, —N═CR^{c}R^{c}, —C(NR^{g})OR^{c}, —C(NR^{g})NR^{c}R^{c}, —C(NR^{g})SR^{c}, —C(NR^{g})NR^{g}NR^{c}R^{c}, —C(NOR^{g})R^{c}, —C(NOR^{g})NR^{c}R^{c}, —C(NNR^{g}R^{g})R^{c}, —C[NNR^{g}C(O)NR^{g}R^{g}]R^{c}, —OS(O)R^{c}, —OS(O)OR^{c}, —OS(O)NR^{c}R^{c}, —OS(O)₂R^{c}, —OS(O)₂OR^{c}, —OS(O)₂NR^{c}R^{c}, —OC(O)R^{c}, —OC(O)OR^{c}, —OC(O)SR^{c}, —OC(O)NR^{c}R^{c}, —O[C(O)]₂NR^{c}R^{c}, —O[C(O)NR^{g}]₂NR^{c}R^{c}, —OC(S)R^{c}, —OC(NR^{g})R^{c}, —OC(NR^{g})NR^{c}R^{c}, —ONR^{g}C(O)R^{c}, —S(O)R^{c}, —S(O)OR^{c}, —S(O)NR^{c}R^{c}, —S(O)₂R^{c}, —S(O)₂OR^{c}, —S(O)₂NR^{c}R^{c}, —[S(O)₂]₂NR^{c}R^{c}, —SC(O)R^{c}, —SC(O)OR^{c}, —SC(O)NR^{c}R^{c}, —SC(S)R^{c}, —SC(NR^{g})R^{c}, —SC(NR^{g})NR^{c}R^{c}, —NR^{g}C(O)R^{c}, —NR^{g}C(O)OR^{c}, —NR^{g}C(O)NR^{c}R^{c}, —NR^{g}C(O)SR^{c}, —NR^{g}C(O)NR^{g}NR^{c}R^{c}, —NR^{g}C(S)R^{c}, —NR^{g}C(S)NR^{c}R^{c}, —NR^{g}C(NR^{g})R^{c}, —N═CR^{c}NR^{c}R^{c}, —NR^{g}C(NR^{g})OR^{c}, —NR^{g}C(NR^{g})NR^{c}R^{c}, —NR^{g}C(NR^{g})SR^{c}, —NR^{g}C(NOR^{g})R^{c}, —NR^{g}S(O)R^{c}, —NR^{g}S(O)OR^{c}, —NR^{g}S(O)₂R^{c}, —NR^{g}S(O)₂OR^{c}, —NR^{g}S(O)₂NR^{c}R^{c}, —NR^{g}NR^{g}C(O)R^{c}, —NR^{g}NR^{g}C(O)NR^{c}R^{c}, —NR^{g}NR^{g}C(NR^{g})R^{c}, —NR^{g}[C(O)]₂R^{c}, —NR^{g}[C(O)]₂OR^{c}, —NR^{g}[C(O)]₂NR^{c}R^{c}, —[NR^{g}C(O)]₂R^{c}, —[NR^{g}C(O)]₂OR^{c}, —NR^{g}[S(O)₂]₂R^{c}, —N(OR^{g})C(O)R^{c}, —N[C(O)R^{c}]NR^{c}R^{c}, —N[C(O)R^{c}]₂, —N[S(O)₂R^{c]}₂, —N{[C(O)]₂R^{c}}₂, —N{[C(O)]₂OR^{c}}₂ and —N{[C(O)]₂NR^{c}R^{c}}₂ and the bivalent substituents ═O, ═S, ═NR^{g}, ═NOR^{g}, ═NNR^{g}R^{g} and ═NNR^{g}C(O)NR^{g}R^{g}, while these bivalent substituents may only be substituents in non-aromatic ring systems;

each R^{c} independently denotes hydrogen or a group optionally substituted by one or more identical or different R^{d} and/or R^{e}, selected from among C₁₋₆alkyl, 2-6 membered heteroalkyl, C₁₋₆haloalkyl, C₃₋₁₀cycloalkyl, C₄₋₁₆cycloalkylalkyl, C₆₋₁₀aryl, C₇₋₁₆arylalkyl, 5-12 membered hetero-aryl, 6-18 membered heteroarylalkyl, 3-14 membered heterocycloalkyl and 4-14 membered heterocycloalkylalkyl;

each R^{d} denotes a suitable substituent and is independently selected from among —OR^{e}, —SR^{e}, —NR^{e}R^{e}, —ONR^{e}R^{e}, —N(OR^{e})R^{e}, —N(R^{g})NR^{e}R^{e}, halogen, —CN, —NC, —OCN, —SCN, —NO, —NO₂, —N₃, —C(O)R^{e}, —C(O)OR^{e}, —C(O)NR^{e}R^{e}, —C(O)SR^{e}, —C(O)NR^{g}NR^{e}R^{e}, —C(O)NR^{g}OR^{e}, —[C(O)]₂NR^{e}R^{e}, —[C(O)NR^{g}]₂R^{e}, —C(S)R^{e}, —C(S)OR^{e}, —C(S)NR^{e}R^{e}, —C(S)SR^{e}, —C(NR^{g})R^{e}, —N═CR^{e}R^{e}, —C(NR^{g})OR^{e}, —C(NR^{g})NR^{e}R^{e}, —C(NR^{g})SR^{e}, —C(NR^{g})NR^{g}NR^{e}R^{e}, —C(NOR^{g})R^{e}, —C(NOR^{g})NR^{e}R^{e}, —C(NNR^{g}R^{g})R^{e}, —C[NNR^{g}C(O)NR^{g}R^{g}]R^{e}, —OS(O)R^{e}, —OS(O)OR^{e}, —OS(O)NR^{e}R^{e}, —OS(O)₂R^{e}, —OS(O)₂OR^{e}, —OS(O)₂NR^{e}R^{e}, —OC(O)R^{e}, —OC(O)OR^{e}, —OC(O)SR^{e}, —OC(O)NR^{e}R^{e}, —O[C(O)]₂NR^{e}R^{e}, —O[C(O)NR^{g}]₂NR^{e}R^{e}, —OC(S)R^{e}, —OC(NR^{g})R^{e}, —OC(NR^{g})NR^{e}R^{e}, —ONR^{g}C(O)R^{e}, —S(O)R^{e}, —S(O)OR^{e}, —S(O)NR^{e}R^{e}, —S(O)₂R^{e}, —S(O)₂OR^{e}, —S(O)₂NR^{e}R^{e}, —[S(O)₂]₂NR^{e}R^{e}, —SC(O)R^{e}, —SC(O)OR^{e}, —SC(O)NR^{e}R^{e}, —SC(S)R^{e}, —SC(NR^{g})R^{e}, —SC(NR^{g})NR^{e}R^{e}, —NR^{g}C(O)R^{e}, —NR^{g}C(O)OR^{e}, —NR^{g}C(O)NR^{e}R^{e}, —NR^{g}C(O)SR^{e}, —NR^{g}C(O)NR^{g}NR^{e}R^{e}, —NR^{g}C(S)R^{e}, —NR^{g}C(S)NR^{e}R^{e}, —NR^{g}C(NR^{g})R^{e}, —N═CR^{e}NR^{e}R^{e}, —NR^{g}C(NR^{g})OR^{e}, —NR^{g}C(NR^{g})NR^{e}R^{e}, —NR^{g}C(NR^{g})SR^{e}, —NR^{g}C(NOR^{g})R^{e}, —NR^{g}S(O)R^{e}, —NR^{g}S(O)OR^{e}, —NR^{g}S(O)₂R^{e}, —NR^{g}S(O)₂OR^{e}, —NR^{g}S(O)₂NR^{e}R^{e}, —NR^{g}NR^{g}C(O)R^{e}, —NR^{g}NR^{g}C(O)NR^{e}R^{e}, —NR^{g}NR^{g}C(NR^{g})R^{e}, —NR^{g}[C(O)]₂R^{e}, —NR^{g}[C(O)]₂OR^{e}, —NR^{g}[C(O)]₂NR^{e}R^{e}, —[NR^{g}C(O)]₂R^{e}, —[NR^{g}C(O)]₂OR^{e}, —NR^{g}[S(O)₂]₂R^{e}, —N(OR^{g})C(O)R^{e}, —N[C(O)R^{e}]NR^{e}R^{e}, —N[C(O)R^{e}]₂, —N[S(O)₂R^{e}]₂, —N{[C(O)]₂R^{e}}₂, —N{[C(O)]₂OR^{e}}₂ and —N{[C(O)]₂NR^{e}R^{e}}₂ and the bivalent substituents ═O, ═S, ═NR^{g}, ═NOR^{g}, ═NNR^{g}R^{g} and ═NNR^{g}C(O)NR^{g}R^{g}, while these bivalent substituents may only be substituents in non-aromatic ring systems;

each R^{e} independently denotes hydrogen or a group optionally substituted by one or more identical or different R^{f} and/or R^{g}, selected from among C₁₋₆alkyl, 2-6 membered heteroalkyl, C₁₋₆haloalkyl, C₃₋₁₀cycloalkyl, C₄₋₁₆cycloalkylalkyl, C₆₋₁₀aryl, C₇₋₁₆arylalkyl, 5-12 membered heteroaryl, 6-18 membered heteroarylalkyl, 3-14 membered heterocycloalkyl and 4-14 membered heterocycloalkylalkyl;

each R^{f} denotes a suitable substituent and is independently selected from among —OR^{g}, —SR^{g}, —NR^{g}R^{g}, —ONR^{g}R^{g}, —N(OR^{g})R^{g}, —N(R^{h})NR^{g}R^{g}, halogen, —CN, —NC, —OCN, —SCN, —NO, —NO₂, —N₃, —C(O)R^{g}, —C(O)OR^{g}, —C(O)NR^{g}R^{g}, —C(O)SR^{g}, —C(O)NR^{h}NR^{g}R^{g}, —C(O)NR^{h}OR^{g}, —[C(O)]₂NR^{g}R^{g}, —[C(O)NR^{h}]₂R^{g}, —C(S)R^{g}, —C(S)OR^{g}, —C(S)NR^{g}R^{g}, —C(S)SR^{g}, —C(NR^{h})R^{g}, —N═CR^{g}R^{g}, —C(NR^{h})OR^{g}, —C(NR^{h})NR^{g}R^{g}, —C(NR^{h})SR^{g}, —C(NR^{h})NR^{h}NR^{g}R^{g}, —C(NOR^{h})R^{g}, —C(NOR^{h})NR^{g}R^{g}, —C(NNR^{h}R^{h})R^{g}, —C[NNR^{h}C(O)NR^{h}R^{h}]R^{g}, —OS(O)R^{g}, —OS(O)OR^{g}, —OS(O)NR^{g}R^{g}, —OS(O)₂R^{g}, —OS(O)₂OR^{g}, —OS(O)₂NR^{g}R^{g}, —OC(O)R^{g}, —OC(O)OR^{g}, —OC(O)SR^{g}, —OC(O)NR^{g}R^{g}, —O[C(O)]₂NR^{g}R^{g}, —O[C(O)NR^{h}]₂NR^{g}R^{g}, —OC(S)R^{g}, —OC(NR^{h})R^{g}, —OC(NR^{h})NR^{g}R^{g}, —ONR^{h}C(O)R^{g}, —S(O)R^{g}, —S(O)OR^{g}, —S(O)NR^{g}R^{g}, —S(O)₂R^{g}, —S(O)₂OR^{g}, —S(O)₂NR^{g}R^{g}, —[S(O)₂]₂NR^{g}R^{g}, —SC(O)R^{g}, —SC(O)OR^{g}, —SC(O)NR^{g}R^{g}, —SC(S)R^{g}, —SC(NR^{h})R^{g}, —SC(NR^{h})NR^{g}R^{g}, —NR^{h}C(O)R^{g}, —NR^{h}C(O)OR^{g}, —NR^{h}C(O)NR^{g}R^{g}, —NR^{h}C(O)SR^{g}, —NR^{h}C(O)NR^{h}NR^{g}R^{g}, —NR^{h}C(S)R^{g}, —NR^{h}C(S)NR^{g}R^{g}, —NR^{h}C(NR^{h})R^{g}, —N═CR^{g}NR^{g}R^{g}, —NR^{h}C(NR^{h})OR^{g}, —NR^{h}C(NR^{h})NR^{g}R^{g}, —NR^{h}C(NR^{h})SR^{g}, —NR^{h}C(NOR^{h})R^{g}, —NR^{h}S(O)R^{g}, —NR^{h}S(O)OR^{g}, —NR^{h}S(O)₂R^{g}, —NR^{h}S(O)₂OR^{g}, —NR^{h}S(O)₂NR^{g}R^{g}, —NR^{h}NR^{h}C(O)R^{g}, —NR^{h}NR^{h}C(O)NR^{g}R^{g}, —NR^{h}NR^{h}C(NR^{h})R^{g}, —NR^{h}[C(O)]₂R^{g}, —NR^{h}[C(O)]₂OR^{g}, —NR^{h}[C(O)]₂NR^{g}R^{g}, —[NR^{h}C(O)]₂R^{g}, —[NR^{h}C(O)]₂OR^{g}, —NR^{h}[S(O)₂]₂R^{g}, —N(OR^{h})C(O)R^{g}, —N[C(O)R^{g}]NR^{g}R^{g}, —N[C(O)R^{g}]₂, —N[S(O)₂R^{g}]₂, —N{[C(O)]₂R^{g}}₂, —N{[C(O)]₂OR^{g}}₂ and —N{[C(O)]₂NR^{g}R^{g}}₂ and the bivalent substituents ═0, ═S, ═NR^{h}, ═NOR^{h}, ═NNR^{h}R^{h} and ═NNR^{h}C(O)NR^{h}R^{h}, while these bivalent substituents may only be substituents in non-aromatic ring systems;

each R^{g} independently denotes hydrogen or a group optionally substituted by one or more identical or different R^{h}, selected from among C₁₋₆alkyl, 2-6 membered heteroalkyl, C₁₋₆haloalkyl, C₃₋₁₀cycloalkyl, C₄₋₁₆cycloalkylalkyl, C₆₋₁₀aryl, C₇₋₁₆arylalkyl, 5-12 membered heteroaryl, 6-18 membered heteroarylalkyl, 3-14 membered heterocycloalkyl and 4-14 membered heterocycloalkylalkyl;

each R^{h} is independently selected from among hydrogen, C₁₋₆alkyl, 2-6 membered heteroalkyl, C₁₋₆haloalkyl, C₃₋₁₀cycloalkyl, C₄₋₁₆cycloalkylalkyl, C₆₋₁₀aryl, C₇₋₁₆arylalkyl, 5-12 membered heteroaryl, 6-18 membered heteroarylalkyl, 3-14 membered heterocycloalkyl and 4-14 membered heterocycloalkylalkyl;

with the proviso that
N-[2-amino-5-(5-isoxazol-3-yl-thiophen-2-yl)-phenyl]-4-methoxy-benzamide and
N-{3-[2-(5-carbamimidoyl-2-methylsulphanyl-thiophen-3-yl)-thiazol-4-yl]-phenyl}-4-fluoro-benzamide
   are excluded,

while the compounds (1) may optionally also be present in the form of the tautomers, the racemates, the enantiomers, the diastereomers, the mixtures thereof and the polymorphs thereof or as pharmacologically acceptable salts of all the above-mentioned forms.

In one aspect (A1) the invention relates to compounds wherein

L is selected from among —C(O)NH— and —NHC(O)—.

In another aspect (B1) the invention relates to compounds wherein

neither R³ nor R⁴ denotes —OH or —NH₂.

In another aspect (B2) the invention relates to compounds wherein

n has the value 0.

In another aspect (C1) the invention relates to compounds wherein

the heteroaromatic five-membered ring in the partial structures (i)-(iv) is not pyrazole.

In another aspect (C2) the invention relates to compounds wherein

the heteroaromatic five-membered ring in the partial structures (i)-(iv) is not imidazole.

In another aspect (C3) the invention relates to compounds wherein

the heteroaromatic five-membered ring in the partial structures (ii)-(iv) is not thiophene.

In another aspect (C4) the invention relates to compounds wherein

the heteroaromatic five-membered ring in the partial structures (i)-(iv) comprises at least two heteroatoms.

In another aspect (C5) the invention relates to compounds wherein

Q has a partial structure selected from the partial structures (v)-(xiii)

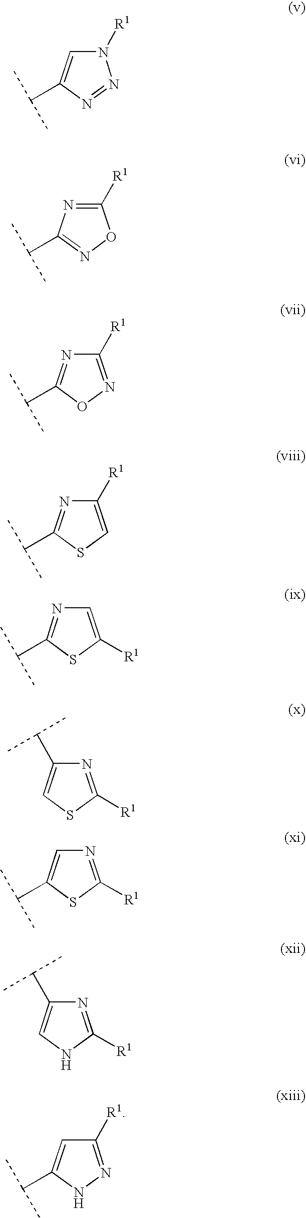

In another aspect (C6) the invention relates to compounds wherein

Q has the partial structure (v)

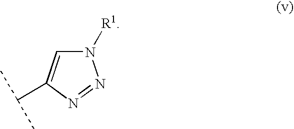

In another aspect (D1) the invention relates to compounds wherein

R¹ denotes a group optionally substituted by one or more identical or different R^{b1} and/or R^{c1}, selected from among C₃₋₁₀cycloalkyl, C₄₋₁₆cycloalkylalkyl, C₇₋₁₆arylalkyl, 5-12 membered heteroaryl, 6-18 membered heteroarylalkyl and 3-14 membered hetero cycloalkyl;

each R^{b1} denotes a suitable substituent and is independently selected from among —OR^{c1}, —SR^{c1}, —NR^{c1}R^{c1}, halogen, —CN, —NO₂, —C(O)R^{c1}, —C(O)OR^{c1}, —C(O)NR^{c1}R^{c1}, —NHC(O)R^{c1}, —NHC(O)OR^{c1}, —NHC(O)NR^{c1}R^{c1}, —S(O)R^{c1}, —S(O)₂R^{c1} as well as the bivalent substituent ═O, while this bivalent substituent may only be a substituent in non-aromatic ring systems;

each R^{c1} independently denotes hydrogen or a group optionally substituted by one or more identical or different R^{d1} and/or R^{e1}, selected from among C₁₋₆alkyl, 2-6 membered heteroalkyl, C₁₋₆haloalkyl, C₃₋₁₀cycloalkyl, C₄₋₁₆cycloalkylalkyl, C₆₋₁₀aryl, C₇₋₁₆arylalkyl, 5-12 membered heteroaryl, 6-18 membered heteroarylalkyl, 3-14 membered heterocycloalkyl and 4-14 membered heterocycloalkylalkyl;

each R^{d1} denotes a suitable substituent and is independently selected from among —OR^{e1}, —NR^{e1}R^{e1}, halogen, —CN, —NO₂, —C(O)R^{e1}, —C(O)OR^{e1}, —C(O)NR^{e1}R^{e1}, —OC(O)R^{e1}, —OC(O)OR^{e1}, —OC(O)NR^{e1}R^{e1}, —NHC(O)R^{e1}, —NHC(O)OR^{e1}, —NHC(O)NR^{e1}R^{e1} as well as the bivalent substituent ═O, while this bivalent substituent may only be a substituent in non-aromatic ring systems;

each R^{e1} is independently selected from among hydrogen, C₁₋₆alkyl, 2-6 membered heteroalkyl, C₁₋₆haloalkyl, C₃₋₁₀cycloalkyl, C₄₋₁₆cycloalkylalkyl, C₆₋₁₀aryl, C₇₋₁₆arylalkyl, 5-12 membered heteroaryl, 6-18 membered heteroarylalkyl, 3-14 membered heterocycloalkyl and 4-14 membered heterocycloalkylalkyl.

In another aspect (D2) the invention relates to compounds wherein

R¹ is a group optionally substituted by one or more identical or different R^{b1} and/or R^{c1}, selected from among 5-12 membered heteroaryl and 3-14 membered heterocycloalkyl.

In another aspect (D3) the invention relates to compounds wherein

R¹ is a group optionally substituted by one or more identical or different R^{b} and/or R^{c}, selected from among 5-12 membered heteroaryl and 3-14 membered heterocycloalkyl.

In another aspect (D4) the invention relates to compounds wherein

R¹ is selected from among pyridyl, pyrimidyl, thiazolyl, imidazolyl, triazolyl, pyrazolyl, pyrrolyl, furanyl, phenyl, benzyl, imidazo[2.1-b]thiazolyl and imidazo[1,2-c]pyridyl and all the above-mentioned ring systems are optionally mono- or polysubstituted.

In another aspect (E1) the invention relates to compounds wherein

R² is selected from among phenyl, pyridyl, pyrazolyl, isoxazolyl, thiazolyl, imidazolyl and oxazolyl, all the above-mentioned groups are optionally substituted by one or more identical or different R^{5b}.

In another aspect (E2) the invention relates to compounds wherein

each R^{5b} is independently selected from among R^{a2} and R^{b2};

each R^{a2} is a group optionally substituted by one or more identical or different R^{b2} and/or R^{c2}, selected from among C₁₋₆alkyl, 2-6 membered heteroalkyl, C₁₋₆haloalkyl, C₃₋₁₀cycloalkyl, C₄₋₁₆cycloalkylalkyl, C₆₋₁₀aryl, C₇₋₁₆arylalkyl, 5-12 membered heteroaryl, 6-18 membered heteroarylalkyl, 3-14 membered heterocycloalkyl and 4-14 membered heterocycloalkylalkyl;

each R^{b2} denotes a suitable substituent and is independently selected from among —OR'², —SR^{c2}, —NR^{c2}R^{c2}, halogen, —CF₃, —CN, —NO₂, —S(O)R^{c2}, —S(O)₂R^{c2}, —S(O)NR^{c2}R^{c2}, —S(O)₂NR^{c2}R^{c2}, —C(O)R^{a2}, —C(O)OR^{c2}, —C(O)NR^{c2}R^{c2}, —OC(O)R^{a2}, —OC(O)OR^{c2}, —OC(O)NR^{c2}R^{c2}, —NHC(O)R^{c2}, —NHS(O)₂R^{c2}, —NHC(O)OR^{c2}, —NHC(O)NR^{c2}R^{c2}, —N═CR^{c2}R^{c2}, —N═CR^{c2}NR^{c2}R^{c2} as well as the bivalent substituent ═O, while this bivalent substituent may only be a substituent in non-aromatic ring systems;

each R^{c2} independently denotes hydrogen or a group optionally substituted by one or more identical or different R^{d2} and/or R^{e2}, selected from among C₁₋₆alkyl, 2-6 membered heteroalkyl, C₁₋₆haloalkyl, C₃₋₁₀cycloalkyl, C₄₋₁₆cycloalkylalkyl, C₆₋₁₀aryl, C₇₋₁₆arylalkyl, 5-12 membered heteroaryl, 6-18 membered heteroarylalkyl, 3-14 membered heterocycloalkyl and 4-14 membered heterocycloalkylalkyl;

each R^{d2} denotes a suitable substituent and is independently selected from among —OR^{e2}, —NR^{e2}R^{e2}, halogen, —CN, —NO₂, —C(O)R^{e2}, —C(O)OR^{e2}, —C(O)NR^{e2}R^{e2}, —OC(O)R^{e2}, —OC(O)OR^{e2}, —OC(O)NR^{e2}R^{e2}, —NHC(O)R^{e2}, —NHC(O)OR^{e2}, —NHC(O)NR^{e2}R^{e2} as well as the bivalent substituent ═O, while this bivalent substituent may only be a substituent in non-aromatic ring systems;

each R^{e2} is independently selected from among hydrogen, C₁₋₆alkyl, 2-6 membered heteroalkyl, C₁₋₆haloalkyl, C₃₋₁₀cycloalkyl, C₄₋₁₆cycloalkylalkyl, C₆₋₁₀aryl, C₇₋₁₆arylalkyl, 5-12 membered heteroaryl, 6-18 membered heteroarylalkyl, 3-14 membered heterocycloalkyl and 4-14 membered heterocycloalkylalkyl.

All the above-mentioned structural aspects relating to different molecular parts of the compounds according to the invention (1) may be combined with one another in any desired manner, thus yielding preferred compounds (1). The invention expressly includes all the combinations of the aspects A1, B1 and B2, C1-C6, D1-D4 and E1 and E2 with one another.

In another aspect the invention relates to compounds—or the pharmacologically acceptable salts thereof—of general formula (1) as pharmaceutical compositions.

In another aspect the invention relates to pharmaceutical preparations, containing as active substance one or more compounds of general formula (1) or the pharmacologically acceptable salts thereof optionally in combination with conventional excipients and/or carriers.

In another aspect the invention relates to the use of compounds of general formula (1) for preparing a pharmaceutical composition for the treatment and/or prevention of cancer, infections, inflammations and autoimmune diseases.

In another aspect the invention relates to a pharmaceutical preparation comprising a compound of general formula (1), wherein the compounds (1) may optionally also be present in the form of the tautomers, the racemates, the enantiomers, the diastereomers, the mixtures thereof, the polymorphs thereof or in the form of the pharmacologically acceptable salts of all the above-mentioned forms, and at least one other cytostatic or cytotoxic active substance, different from formula (1).

### DEFINITIONS

As used herein, the following definitions apply, unless stated otherwise:

The use of the prefix C_{x-y}, wherein x and y each represent a natural number (x<y), indicates that the chain or ring structure or combination of chain and ring structure thus designated and mentioned in direct connection may consist of a total of not more than y and not less than x carbon atoms.

The indication of the number of members in groups that contain one or more heteroatom(s) (heteroalkyl, heteroaryl, heteroarylalkyl, heterocycloalkyl, heterocycloalkylalkyl) refers to the total number of atoms of all the ring members or the total of all the ring and chain members.

Alkyl is made up of the sub-groups saturated hydrocarbon chains and unsaturated hydrocarbon chains, while the latter may be further subdivided into hydrocarbon chains with a double bond (alkenyl) and hydrocarbon chains with a triple bond (alkynyl). Alkenyl contains at least one double bond, alkynyl at least one triple bond. If a hydrocarbon chain should have both at least one double bond and at least one triple bond, by definition it belongs to the alkynyl sub-group. All the above-mentioned sub-groups may be further subdivided into straight-chain (unbranched) and branched. If an alkyl is substituted, it may be mono- or polysubstituted independently of one another at all the hydrogen-carrying carbon atoms.

Examples of individual sub-groups are listed below.

Straight-Chain (Unbranched) or Branched, Saturated Hydrocarbon Chains:

methyl; ethyl; n-propyl; isopropyl (1-methylethyl); n-butyl; 1-methylpropyl; isobutyl (2-methylpropyl); sec.-butyl (1-methylpropyl); tent.-butyl (1.1-dimethylethyl); n-pentyl; 1-methylbutyl; 1-ethylpropyl; isopentyl (3-methylbutyl); neopentyl (2,2-dimethyl-propyl); n-hexyl; 2,3-dimethylbutyl; 2,2-dimethylbutyl; 3,3-dimethylbutyl; 2-methyl-pentyl; 3-methylpentyl; n-heptyl; 2-methylhexyl; 3-methylhexyl; 2,2-dimethylpentyl; 2,3-dimethylpentyl; 2,4-dimethylpentyl; 3,3-dimethylpentyl; 2,2,3-trimethylbutyl; 3-ethylpentyl; n-octyl; n-nonyl; n-decyl etc.

Straight-Chained (Unbranched) or Branched Alkenyl:

vinyl (ethenyl); prop-1-enyl; allyl (prop-2-enyl); isopropenyl; but-1-enyl; but-2-enyl; but-3-enyl; 2-methyl-prop-2-enyl; 2-methyl-prop-1-enyl; 1-methyl-prop-2-enyl; 1-methyl-prop-1-enyl; 1-methylidenepropyl; pent-1-enyl; pent-2-enyl; pent-3-enyl; pent-4-enyl; 3-methyl-but-3-enyl; 3-methyl-but-2-enyl; 3-methyl-but-1-enyl; hex-1-enyl; hex-2-enyl; hex-3-enyl; hex-4-enyl; hex-5-enyl; 2,3-dimethyl-but-3-enyl; 2,3-dimethyl-but-2-enyl; 2-methylidene-3-methylbutyl; 2,3-dimethyl-but-1-enyl; hexa-1,3-dienyl; hexa-1,4-dienyl; penta-1,4-dienyl; penta-1,3-dienyl; buta-1,3-dienyl; 2,3-dimethylbuta-1,3-diene etc.

Straight-Chain (Unbranched) or Branched Alkenyl:

ethynyl; prop-1-ynyl; prop-2-ynyl; but-1-ynyl; but-2-ynyl; but-3-ynyl; 1-methyl-prop-2-ynyl etc.

By the terms propyl, butyl, pentyl, hexyl, heptyl, octyl, nonyl, decyl etc. unless otherwise stated are meant saturated hydrocarbon groups with the corresponding number of carbon atoms, including all the isomeric forms.

By the terms propenyl, butenyl, pentenyl, hexenyl, heptenyl, octenyl, nonenyl, decenyl etc. unless otherwise stated are meant unsaturated hydrocarbon groups with the corresponding number of carbon atoms and a double bond, including all the isomeric forms, also (Z)/(E)-isomers, where applicable.

By the terms butadienyl, pentadienyl, hexadienyl, heptadienyl, octadienyl, nonadienyl, decadienyl etc. unless otherwise stated are meant unsaturated hydrocarbon groups with the corresponding number of carbon atoms and two double bonds, including all the isomeric forms, also (Z)/(E)-isomers, where applicable.

By the terms propynyl, butynyl, pentynyl, hexynyl, heptynyl, octynyl, nonynyl, decynyl etc. unless otherwise stated are meant unsaturated hydrocarbon groups with the corresponding number of carbon atoms and a triple bond, including all the isomeric forms.

By the term heteroalkyl are meant groups which are derived from the alkyl as herein-before defined in its widest sense by replacing, in the hydrocarbon chains, one or more of the groups —CH₃ independently of one another by the groups —OH, —SH or —NH₂, one or more of the groups —CH₂— independently of one another by the groups —O—, —S— or —NH—, one or more of the groups

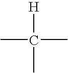

by the group

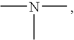

one or more of the groups ═CH— by the group ═N—, one or more of the groups ═CH₂ by the group ═NH or one or more of the groups ≡CH by the group ≡N, while a total of not more than three heteroatoms may be present in one heteroalkyl, there must be at least one carbon atom between two oxygen atoms and between two sulphur atoms or between one oxygen and one sulphur atom and the group as a whole must have chemical stability.

A direct result of the indirect definition/derivation from alkyl is that heteroalkyl is made up of the sub-groups saturated hydrocarbon chains with heteroatom(s), heteroalkenyl and heteroalkynyl, and it may be further subdivided into straight-chain (unbranched) and branched. If a heteroalkyl is substituted, it may be mono- or polysubstituted independently of one another at all the hydrogen-carrying oxygen, sulphur, nitrogen and/or carbon atoms. Heteroalkyl itself as a substituent may be attached to the molecule both through a carbon atom and through a heteroatom.

The following are listed by way of example:

dimethylaminomethyl; dimethylaminoethyl (1-dimethylaminoethyl; 2-dimethyl-aminoethyl); dimethylaminopropyl (1-dimethylaminopropyl, 2-dimethylaminopropyl, 3-dimethylaminopropyl); diethylaminomethyl; diethylaminoethyl (1-diethylaminoethyl, 2-diethylaminoethyl); diethylaminopropyl (1-diethylaminopropyl, 2-diethylamino-propyl, 3-diethylaminopropyl); diisopropylaminoethyl (1-diisopropylaminoethyl, 2-di-isopropylaminoethyl); bis-2-methoxyethylamino; [2-(dimethylamino-ethyl)-ethyl-amino]-methyl; 3-[2-(dimethylamino-ethyl)-ethyl-amino]-propyl; hydroxymethyl; 2-hydroxy-ethyl; 3-hydroxypropyl; methoxy; ethoxy; propoxy; methoxymethyl; 2-methoxyethyl etc.

Haloalkyl is derived from alkyl as hereinbefore defined in its broadest sense, by replacing one or more hydrogen atoms of the hydrocarbon chain independently of one another by halogen atoms, which may be identical or different. A direct result of the indirect definition/derivation from alkyl is that haloalkyl is made up of the sub-groups saturated hydrohalogen chains, haloalkenyl and haloalkynyl, and it may be further subdivided into straight-chain (unbranched) and branched. If a haloalkyl is substituted, it may be mono- or polysubstituted independently of one another at all the hydrogen-carrying carbon atoms.

The following are listed by way of example:

—CF₃; —CHF₂; —CH₂F; —CF₂CF₃; —CHFCF₃; —CH₂CF₃; —CF₂CH₃; —CHFCH₃; —CF₂CF₂CF₃; —CF₂CH₂CH₃; —CF═CF₂; —CCl═CH₂; —CBr═CH₂; —CI═CH₂; —C≡C—CF₃; —CHFCH₂CH₃; —CHFCH₂CF₃ etc.

Halogen relates to fluorine, chlorine, bromine and/or iodine atoms.

Cycloalkyl is made up of the sub-groups monocyclic hydrocarbon rings, bicyclic hydrocarbon rings and spirohydrocarbon rings, while each sub-group may be further subdivided into saturated and unsaturated (cycloalkenyl). By unsaturated is meant that there is at least one double bond in the ring system, but no aromatic system is formed. In bicyclic hydrocarbon rings two rings are linked such that they share at least two carbon atoms. In spirohydrocarbon rings one carbon atom (spiroatom) is shared by two rings. If a cycloalkyl is substituted, it may be mono- or polysubstituted independently of one another at all the hydrogen-carrying carbon atoms. Cycloalkyl itself as a substituent may be attached to the molecule through any suitable position of the ring system.

The following individual sub-groups are listed by way of example:

Monocyclic Hydrocarbon Rings, Saturated:

cyclopropyl; cyclobutyl; cyclopentyl; cyclohexyl; cycloheptyl etc.

Monocyclic Hydrocarbon Rings, Unsaturated:

cycloprop-1-enyl; cycloprop-2-enyl; cyclobut-1-enyl; cyclobut-2-enyl; cyclopent-1-enyl; cyclopent-2-enyl; cyclopent-3-enyl; cyclohex-1-enyl; cyclohex-2-enyl; cyclohex-3-enyl; cyclohept-1-enyl; cyclohept-2-enyl; cyclohept-3-enyl; cyclohept-4-enyl; cyclobuta-1,3-dienyl; cyclopenta-1,4-dienyl; cyclopenta-1,3-dienyl; cyclopenta-2,4-dienyl; cyclohexa-1,3-dienyl; cyclohexa-1,5-dienyl; cyclohexa-2,4-dienyl; cyclohexa-1,4-dienyl; cyclohexa-2,5-dienyl etc.

Bicyclic Hydrocarbon Rings (Saturated and Unsaturated):

bicyclo[2.2.0]hexyl; bicyclo[3.2.0]heptyl; bicyclo[3.2.1]octyl; bicyclo[2.2.2]octyl; bicyclo[4.3.0]nonyl (octahydroindenyl); bicyclo[4.4.0]decyl (decahydronaphthalene); bicyclo[2.2.1]heptyl (norbornyl); (bicyclo[2.2.1]hepta-2,5-dienyl (norborna-2,5-dienyl); bicyclo[2.2.1]hept-2-enyl (norbornenyl); bicyclo[4.1.0]heptyl (norcaranyl); bicyclo-[3.1.1]heptyl (pinanyl) etc.

Spirohydrocarbon Rings (Saturated and Unsaturated):

spiro[2.5]octyl, spiro[3.3]heptyl, spiro[4.5]dec-2-ene etc.

Cycloalkylalkyl denotes the combination of the alkyl and cycloalkyl groups defined hereinbefore, in each case in their broadest sense. The alkyl group as substituent is directly linked to the molecule and is in turn substituted by a cycloalkyl group. The linking of alkyl and cycloalkyl in both groups may be effected by means of any suitable carbon atoms. The sub-groups of alkyl and cycloalkyl are also included in the combination of the two groups.

Aryl denotes mono-, bi- or tricyclic carbon rings with at least one aromatic ring. If an aryl is substituted, the substitution may be mono- or polysubstitution in each case, at all the hydrogen-carrying carbon atoms, independently of one another. Aryl itself may be linked to the molecule as substituent via any suitable position of the ring system. Typical examples are listed below.

phenyl; naphthyl; indanyl (2,3-dihydroindenyl); 1,2,3,4-tetrahydronaphthyl; fluorenyl etc.

Arylalkyl denotes the combination of the groups alkyl and aryl as hereinbefore defined, in each case in their broadest sense. The alkyl group as substituent is directly linked to the molecule and is in turn substituted by an aryl group. The alkyl and aryl may be linked in both groups via any carbon atoms suitable for this purpose. The respective sub-groups of alkyl and aryl are also included in the combination of the two groups.

Typical examples are listed below:

benzyl; 1-phenylethyl; 2-phenylethyl; phenylvinyl; phenylallyl etc.

Heteroaryl denotes monocyclic aromatic rings or polycyclic rings with at least one aromatic ring, which, compared with corresponding aryl or cycloalkyl, contain instead of one or more carbon atoms one or more identical or different heteroatoms, selected independently of one another from among nitrogen, sulphur and oxygen, while the resulting group must be chemically stable. If a heteroaryl is substituted, the substitution may be mono- or polysubstitution in each case, at all the hydrogen-carrying carbon and/or nitrogen atoms, independently of one another. Heteroaryl itself as substituent may be linked to the molecule via any suitable position of the ring system, both carbon and nitrogen.

Typical examples are listed below.

Monocyclic Heteroaryls:

furyl; thienyl; pyrrolyl; oxazolyl; thiazolyl; isoxazolyl; isothiazolyl; pyrazolyl; imidazolyl; triazolyl; tetrazolyl; oxadiazolyl; thiadiazolyl; pyridyl; pyrimidyl; pyridazinyl; pyrazinyl; triazinyl; pyridyl-N-oxide; pyrrolyl-N-oxide; pyrimidinyl-N-oxide; pyridazinyl-N-oxide; pyrazinyl-N-oxide; imidazolyl-N-oxide; isoxazolyl-N-oxide; oxazolyl-N-oxide; thiazolyl-N-oxide; oxadiazolyl-N-oxide; thiadiazolyl-N-oxide; triazolyl-N-oxide; tetrazolyl-N-oxide etc.

Polycyclic Heteroaryls:

indolyl; isoindolyl; benzofuryl; benzothienyl; benzoxazolyl; benzothiazolyl; benzisoxazolyl; benzisothiazolyl; benzimidazolyl; indazolyl; isoquinolinyl; quinolinyl; quinoxalinyl; cinnolinyl; phthalazinyl; quinazolinyl; benzotriazinyl; indolizinyl; oxazolopyridyl; imidazopyridyl; naphthyridinyl; indolinyl; isochromanyl; chromanyl; tetrahydroisoquinolinyl; isoindolinyl; isobenzotetrahydrofuryl; isobenzotetrahydrothienyl; isobenzothienyl; benzoxazolyl; pyridopyridyl; benzotetrahydrofuryl; benzotetrahydro-thienyl; purinyl; benzodioxolyl; phenoxazinyl; phenothiazinyl; pteridinyl; benzothiazolyl; imidazopyridyl; imidazothiazolyl; dihydrobenzisoxazinyl; benzisoxazinyl; benzoxazinyl; dihydrobenzisothiazinyl; benzopyranyl; benzothiopyranyl; cumarinyl; isocumarinyl; chromonyl; chromanonyl; tetrahydroquinolinyl; dihydroquinolinyl; dihydroquinolinonyl; dihydroisoquinolinonyl; dihydrocumarinyl; dihydroisocumarinyl; isoindolinonyl; benzodioxanyl; benzoxazolinonyl; quinolinyl-N-oxide; indolyl-N-oxide; indolinyl-N-oxide; isoquinolyl-N-oxide; quinazolinyl-N-oxide; quinoxalinyl-N-oxide; phthalazinyl-N-oxide; indolizinyl-N-oxide; indazolyl-N-oxide; benzothiazolyl-N-oxide; benzimidazolyl-N-oxide; benzo-thiopyranyl-S-oxide and benzothiopyranyl-S,S-dioxide etc.

Heteroarylalkyl denotes the combination of the alkyl and heteroaryl groups defined hereinbefore, in each case in their broadest sense. The alkyl group as substituent is directly linked to the molecule and is in turn substituted by a heteroaryl group. The linking of the alkyl and heteroaryl may be achieved on the alkyl side via any carbon atoms suitable for this purpose and on the heteroaryl side by any carbon or nitrogen atoms suitable for this purpose. The respective sub-groups of alkyl and heteroaryl are also included in the combination of the two groups.

By the term heterocycloalkyl are meant groups which are derived from the cycloalkyl as hereinbefore defined if in the hydrocarbon rings one or more of the groups —CH₂— are replaced independently of one another by the groups —O—, —S— or —NH— or one or more of the groups ═CH— are replaced by the group ═N—, while not more than five heteroatoms may be present in total, there must be at least one carbon atom between two oxygen atoms and between two sulphur atoms or between one oxygen and one sulphur atom and the group as a whole must be chemically stable. Heteroatoms may simultaneously be present in all the possible oxidation stages (sulphur→sulphoxide —SO—, sulphone —SO₂—; nitrogen→N-oxide). It is immediately apparent from the indirect definition/derivation from cycloalkyl that heterocycloalkyl is made up of the sub-groups monocyclic hetero-rings, bicyclic hetero-rings and spirohetero-rings, while each sub-group can also be further subdivided into saturated and unsaturated (heterocycloalkenyl). The term unsaturated means that in the ring system in question there is at least one double bond, but no aromatic system is formed. In bicyclic hetero-rings two rings are linked such that they have at least two atoms in common. In spirohetero-rings one carbon atom (spiroatom) is shared by two rings. If a heterocycloalkyl is substituted, the substitution may be mono- or polysubstitution in each case, at all the hydrogen-carrying carbon and/or nitrogen atoms, independently of one another. Heterocycloalkyl itself as substituent may be linked to the molecule via any suitable position of the ring system.

Typical examples of individual sub-groups are listed below.

Monocyclic Heterorings (Saturated and Unsaturated):

tetrahydrofuryl; pyrrolidinyl; pyrrolinyl; imidazolidinyl; thiazolidinyl; imidazolinyl; pyrazolidinyl; pyrazolinyl; piperidinyl; piperazinyl; oxiranyl; aziridinyl; azetidinyl; 1,4-dioxanyl; azepanyl; diazepanyl; morpholinyl; thiomorpholinyl; homomorpholinyl; homopiperidinyl; homopiperazinyl; homothiomorpholinyl; thiomorpholinyl-5-oxide; thiomorpholinyl-S,S-dioxide; 1,3-dioxolanyl; tetrahydropyranyl; tetrahydrothiopyranyl; [1,4]-oxazepanyl; tetrahydrothienyl; homothiomorpholinyl-S,S-dioxide; oxazolidinonyl; dihydropyrazolyl; dihydropyrrolyl; dihydropyrazinyl; dihydropyridyl; dihydro-pyrimidinyl; dihydrofuryl; dihydropyranyl; tetrahydrothienyl-5-oxide; tetrahydrothienyl-S,S-dioxide; homothiomorpholinyl-5-oxide; 2,3-dihydroazet; 2H-pyrrolyl; 4H-pyranyl; 1,4-dihydropyridinyl etc.

Bicyclic Heterorings (Saturated and Unsaturated):

8-azabicyclo [3.2.1]octyl; 8-azabicyclo [5.1.0]octyl; 2-oxa-5-azabicyclo [2.2.1]heptyl; 8-oxa-3-aza-bicyclo [3.2.1]octyl; 3.8-diaza-bicyclo [3.2.1]octyl; 2.5-diaza-bicyclo-[2.2.1]heptyl; 1-aza-bicyclo [2.2.2]octyl; 3.8-diaza-bicyclo [3.2.1]octyl; 3.9-diaza-bicyclo [4.2.1]nonyl; 2.6-diaza-bicyclo [3.2.2]nonyl etc.

Spiro-Heterorings (Saturated and Unsaturated):

1,4-dioxa-spiro[4.5]decyl; 1-oxa-3.8-diaza-spiro[4.5]decyl; and 2,6-diaza-spiro[3.3]heptyl; 2,7-diaza-spiro[4.4]nonyl; 2,6-diaza-spiro[3.4]octyl; 3,9-diaza-spiro[5.5]undecyl; 2,8-diaza-spiro[4.5]decyl etc.

Heterocycloalkylalkyl denotes the combination of the alkyl and heterocycloalkyl groups defined hereinbefore, in each case in their broadest sense. The alkyl group as substituent is directly linked to the molecule and is in turn substituted by a heterocycloalkyl group. The linking of the alkyl and heterocycloalkyl may be achieved on the alkyl side via any carbon atoms suitable for this purpose and on the heterocycloalkyl side by any carbon or nitrogen atoms suitable for this purpose. The respective sub-groups of alkyl and heterocycloalkyl are also included in the combination of the two groups.

The term “substituted” indicates that a hydrogen atom which is bound directly to the atom in question is replaced by another atom or another group of atoms. Alternatively substitution may also take place at an atom if there are free electrons available at this atom. Depending on the starting conditions (number of hydrogen atoms, number of free electrons) there may be mono- or polysubstitution at an atom. Thus, for example a free electron pair may be substituted by two monovalent substituents.

Bivalent substituents such as for example ═S, ═NR, ═NOR, ═NNRR, ═NN(R)C(O)NRR, ═N₂ or the like can only be substituents at carbon atoms, the bivalent substituent ═O may also be a substituent at heteroatoms. Generally speaking, substitution by a bivalent substituent can only take place at non-aromatic ring systems and requires exchanging for two geminal hydrogen atoms, i.e. hydrogen atoms which are bound to the same carbon atom saturated before the substitution, or for a free electron pair. Substitution by a bivalent substituent is therefore only possible at the group —CH₂— or heteroatoms or a non-aromatic ring system.

Additionally, by the term “suitable substituent” is meant a substituent which on the one hand is suitable on account of its valency and on the other hand leads to a system with chemical stability.

Groups or substituents are frequently selected from among a number of alternative groups/substituents with a corresponding group name (e.g. R^{a}, R^{b} etc). If such a group is used repeatedly to define a compound according to the invention in different parts of the molecular, it should always be borne in mind that the respective uses must be viewed totally independently of one another.

### List of Abbreviations

| | |
|---|---|
| abs. | absolute, anhydrous |
| Ac | acetyl |
| Bn | benzyl |
| Boc | tert.-butyloxycarbonyl |
| Bu | butyl |
| c | concentration |
| chex | cyclohexane |
| d | day(s) |
| DBAD | di-tert.-butyl-azodicarboxylate |
| DC, TLC | thin layer chromatography |
| DCM | dichloromethane |
| DEA | diethylamine |
| DIPEA | N-ethyl-N,N-diisopropylamine (Hünig base) |
| DMAP | 4-N,N-dimethylaminopyridine |
| DME | 1,2-dimethoxyethane |
| DMF | N,N-dimethylformamide |
| DMSO | dimethylsulphoxide |
| EE | ethyl acetate |
| eq | equivalent(s) |
| ESI | electron spray ionization |
| Et | ethyl |
| EtOH | ethanol |
| h | hour |
| HATU | O-(7-azabenzotriazol-1-yl)-N,N,N′,N′-tetramethyl- |
| | uronium tetrafluorophosphate |
| hex | hexyl |
| HPLC | high performance liquid chromatography |
| Hünig-base | N-ethyl-N,N-diisopropylamine |
| i | iso |
| IR | infrared spectroscopy |
| cat. | catalyst, catalytic |
| conc. | concentrated |
| b.p. | boiling point |
| LC | liquid chromatography |
| LHMDS | lithium-hexamethyldisilazane |
| soln. | solution |
| Me | methyl |
| MeOH | methanol |
| min | minutes |
| MPLC | medium pressure liquid chromatography |
| MS | mass spectrometry |
| NMP | N-methylpyrrolidone |
| NP | normal phase |
| n.a. | not available |
| Ph | phenyl |
| Pr | propyl |
| PS | polystyrene |
| Py | pyridine |
| rac | racemic |
| R_{f} (Rf) | retention factor |
| RP | reversed phase |
| RT | ambient temperature |
| TBAF | tetrabutylammonium fluoride |
| TBTU | O-(benzotriazol-1-yl)-N,N,N′,N′-tetramethyl-uronium |
| | tetrafluoroborate |
| temp. | temperature |
| tert. | tertiary |
| Tf | triflate |
| TFA | trifluoroacetic acid |
| THF | tetrahydrofuran |
| TMS | trimethylsilyl |
| t_{Ret} | retention time (HPLC) |
| TsOH | para-toluenesulphonic acid |
| UV | ultraviolet |

Features and advantages of the present invention will become apparent from the following detailed Examples, which illustrate the basics of the invention by way of example, without limiting its scope:

Preparation of the Compounds According to the Invention

General

All the reactions are carried out—unless stated otherwise—in commercially obtainable apparatus using methods conventionally used in chemical laboratories. Air- and/or moisture-sensitive starting materials are stored under protective gas and corresponding reactions and manipulations using them are carried out under protective gas (nitrogen or argon).

Microwave reactions are carried out in an Initiator made by Biotage or Explorer made by CEM in sealed containers (preferably 2, 5 or 20 mL), preferably with stirring.

Chromatography

For the preparative medium pressure chromatography (MPLC, normal phase) silica gel is used which is made by Millipore (named: Granula Silica Si-60A 35-70 μm) or C-18 RP-silica gel (RP-phase) made by Macherey Nagel (named: Polygoprep 100-50 C18). The thin layer chromatography is carried out on ready-made silica gel 60 TLC plates on glass (with fluorescence indicator F-254) made by Merck.

The preparative high pressure chromatography (HPLC) is carried out using columns made by Waters (named: XTerra Prep. MS C18, 5 μm, 30×100 mm or XTerra Prep. MS C18, 5 μm, 50×100 mm OBD or Symmetry C18, 5 μm, 19×100 mm or Sunfire C18 OBD, 19×100 mm, 5 μm or Sunfire Prep C 10 μm OBD 50×150 mm or X-Bridge Prep C18 5 μm OBD 19×50 mm), Agilent (named: Zorbax SB-C8 5 μm PrepHT 21.2×50 mm) and Phenomenex (named: Gemini C18 5 μm AXIA 21.2×50 mm or Gemini C18 10 μm 50×150 mm), the analytical HPLC (reaction control) with columns made by Agilent (named: Zorbax SB-C8, 5 μm, 21.2×50 mm or Zorbax SB-C8 3.5 μm 2.1×50 mm) and Phenomenex (named: Gemini C18 3 μm 2×30 mm).

HPLC-Mass Spectroscopy/UV-Spectrometry

The retention times/MS-ESI⁺ for characterising the examples are obtained using an HPLC-MS apparatus (high performance liquid chromatography with mass detector) made by Agilent. Compounds that elute with the injection peak are given the retention time t_{Ret.}=0.0 min.

The apparatus has the following specification:
Column: Waters, Xterra MS C18, 2.5 μm, 2.1×30 mm, Part. No. 186000592
Eluant: A: H₂O with 0.1% HCOOH; B: acetonitrile (HPLC grade)
Detection: MS: Positive and negative mode
Mass range: 120-900 m/z
Fragmentor: 120
Gain EMV: 1; Threshold: 150; Stepsize: 0.25; UV: 254 nm; Bandwide: 1
Injection: Inj. Vol. 5 μL
Separation: Flow 1.10 mL/min
Column temp.: 40° C.
Gradient: 0.00 min: 5% solvent B
   0.00-2.50 min: 5%→95% solvent B
   2.50-2.80 min: 95% solvent B
   2.81-3.10 min: 95%→5% solvent B

In addition, the following apparatus specification is used in some cases:
Column: Waters, Xterra MS C18, 2.5 μm, 2.1×50 mm, Part. No. 186000594
Eluant: A: deion. Water with 0.1% HCOOH; B: acetonitrile with 0.1% HCOOH
Detection: MS: Positive and negative mode
Mass range: 100-1200 m/z
Fragmentor: 70
Gain EMV: Threshold: 1 mAU; Stepsize: 2 nm; UV: 254 nm as well as 230 nm;
   Bandwide: 8
Injection: Standard 1 μL
Flow: 0.6 mL/min
Column temp.: 35° C.
Gradient: 0.00 min: 5% solvent B
   0.00-2.50 min: 5%→95% solvent B
   2.50-4.00 min: 95% solvent B
   4.00-4.50 min: 95%→5% solvent B
   4.50-6.00 min: 95% solvent A

The compounds according to the invention may be prepared by the methods of synthesis described below, with the substituents of the general formulae having the meanings stated hereinbefore. These methods are intended to illustrate the invention without restricting it to their content or limiting the scope of the compounds claimed to these Examples. Where the preparation of the starting compounds is not described, they are commercially obtainable or may be prepared analogously to known compounds or methods described herein. Substances described in the literature are prepared according to the published methods of synthesis.

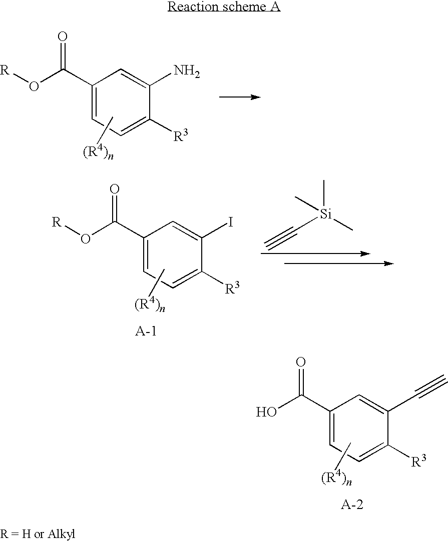

Compounds A-2 may be obtained by various methods. Using methods known from the literature compounds of type A-1 are coupled with TMS-acetylene in a Sonogashira reaction. The cleaving of the silyl group is also carried out using methods known from the literature (e.g. With K₂CO₃ or TBAF). Any exster cleaving is also carried out using methods known from the literature. The compounds A-1 in turn are obtained—if they are not commercially obtainable—by known methods from the corresponding anilines using diazotisation and subsequent reaction with potassium iodide.

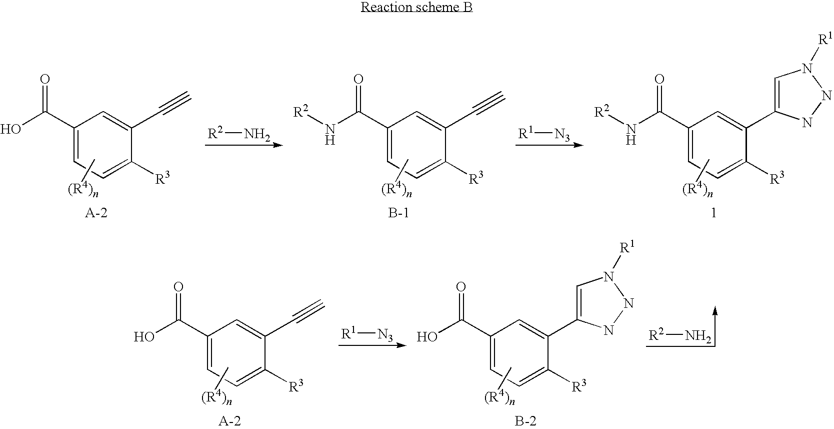

Examples of type 1 are synthesised from the compounds A-2 by an amide coupling reaction (to introduce the group R²) and cyclo addition with an azide (to introduce the group R¹) s, while the two partial steps may be carried out in any desired order. The amide coupling is carried out using methods known from the literature with the aid of common coupling reagents, such as HATU or TBTU, or the compounds A-2 or B-2 are activated by means of thionyl chloride, oxalyl chloride or Ghosez reagent using methods known from the literature to form the corresponding acid chloride and then reacted with the amine R²—NH₂. The amines are commercially obtainable or are synthesised using methods known from the literature. The cycloaddition with the compounds A-2 or B-1 is also carried out using methods known from the literature with the aid of CuSO₄ and sodium ascorbate.

Aryl or heteroaryl azides for introducing the groups R¹ are obtained by known methods from the corresponding amine by diazotisation and reaction with sodium azide. Arylalkyl-azides, heteroarylalkyl-azides and most of the other azides are usually obtained by nucleophilic substitution of the corresponding halides, for example the bromide, with sodium azide.

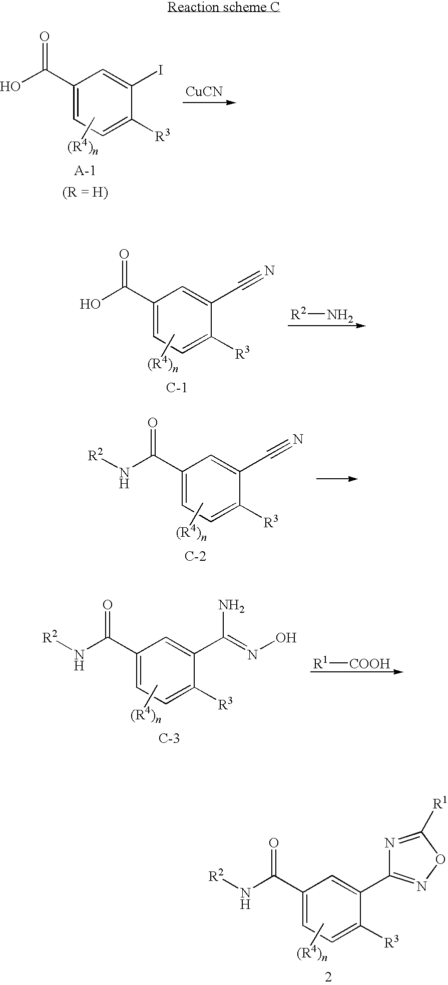

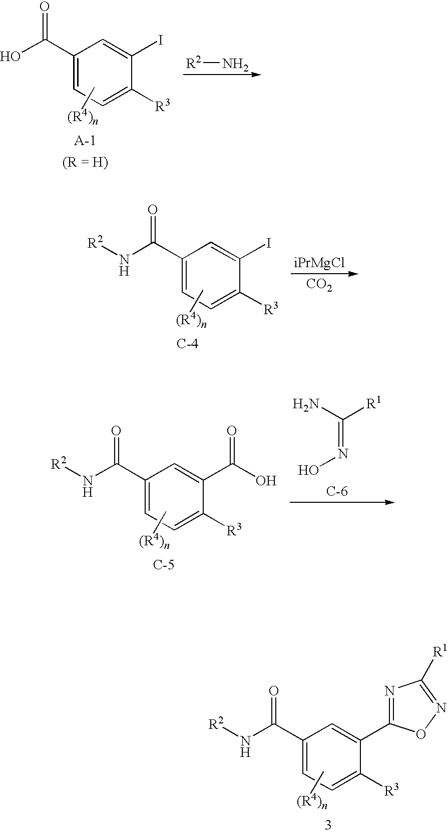

Examples of type 2 are synthesised via the intermediates C-2, which may be obtained starting from A-1 by reacting with CuCN with subsequent amide coupling for introducing the groups R². Reaction of C-2 using methods known from the literature first of all with hydroxylamine zu C-3 and then with activated carboxylic acids yields Examples of type 2. Compounds of type 3 may also be obtained starting from A-1 via the carboxylic acid intermediates C-5, which may be cyclised with hydroxyamidines C-6 using methods known from the literature.

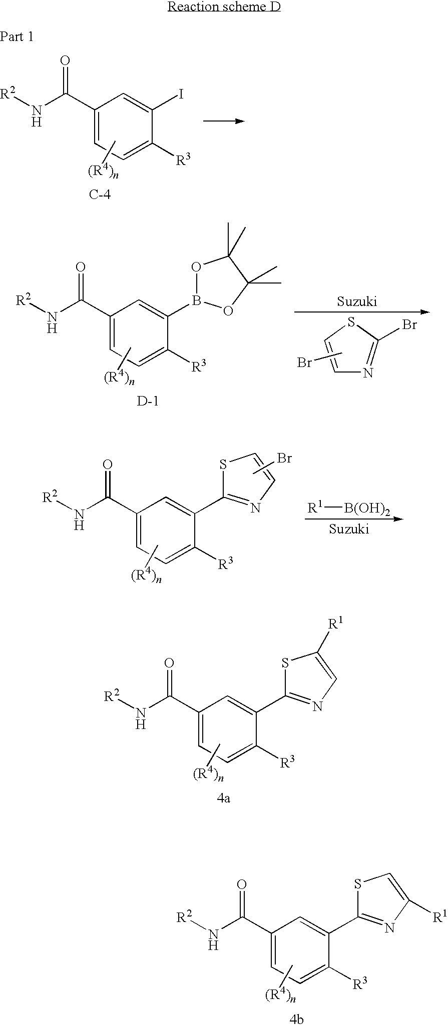

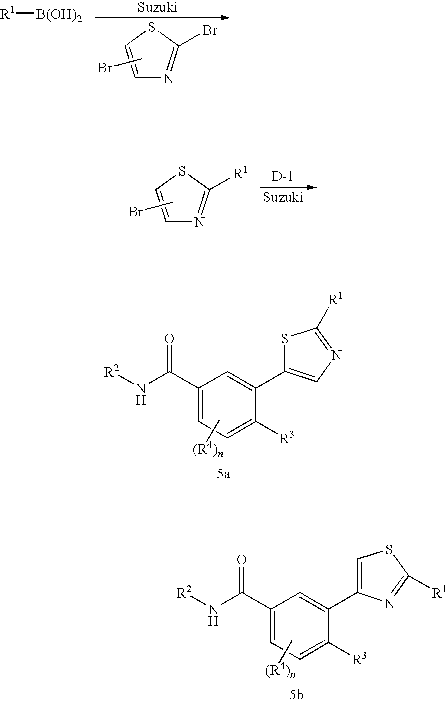

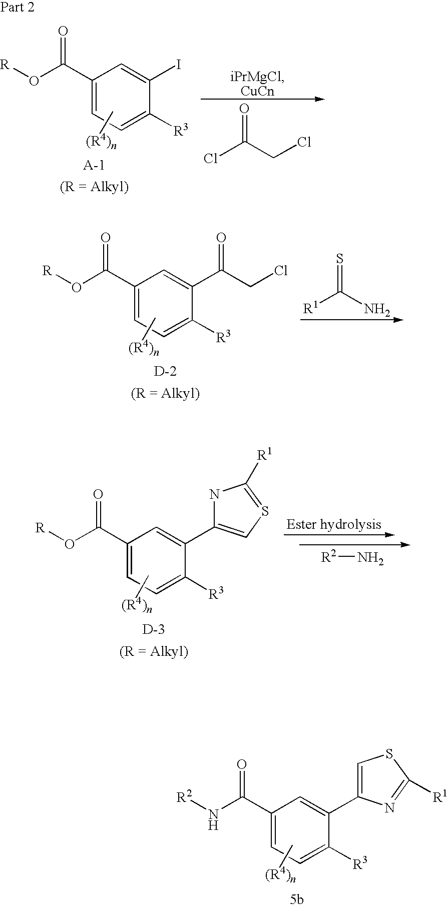

Examples of type 4a, 4b, 5a and 5b are synthesised via the boric acid esters D-1, which are prepared from the intermediates C-4 using methods known from the literature. The target compounds are obtained by two successive Suzuki coupling reactions with commercially obtainable dibromothiazoles, D-1 and R¹—B(OH)₂ using methods known from the literature (reaction scheme D, Part 1). Compounds of type 5b may also be synthesised via the carboxylic acid esters D-3 using methods known from the literature by ester hydrolysis and subsequent amide coupling. The compounds D-3 may in turn be obtained from the α-chloracetophenone derivatives D-2 by reacting with thioamides. The corresponding compounds D-2 are prepared from the intermediates A-1 by halogen-metal exchange and subsequent reaction with a chloroacetic acid chloride (reaction scheme D, Part 2).

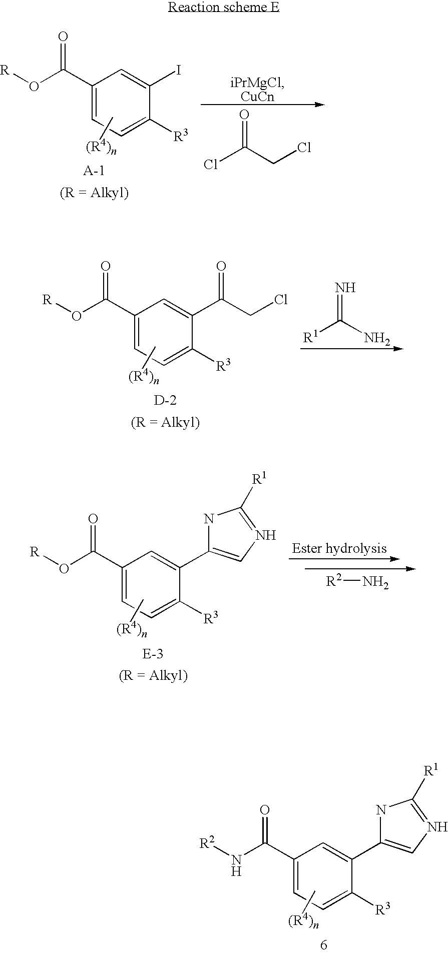

Examples of type 6 are synthesised from the carboxylic acid esters E-3 using methods known from the literature by ester hydrolysis and subsequent amide coupling. The compounds E-3 may in turn be obtained from the α-chloroacetophenone derivatives D-2 by reacting with amidines. The corresponding compounds D-2 are synthesised as described in reaction scheme D.

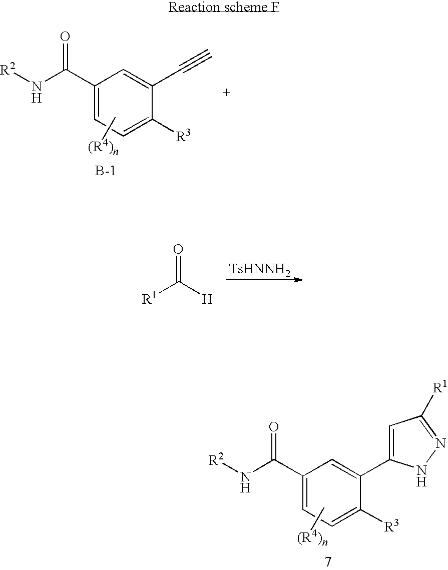

Examples of type 7 are prepared from the intermediates B-1 by reacting with tosylhydrazones, which are generated in situ from the corresponding aldehydes and tosylhydrazine.

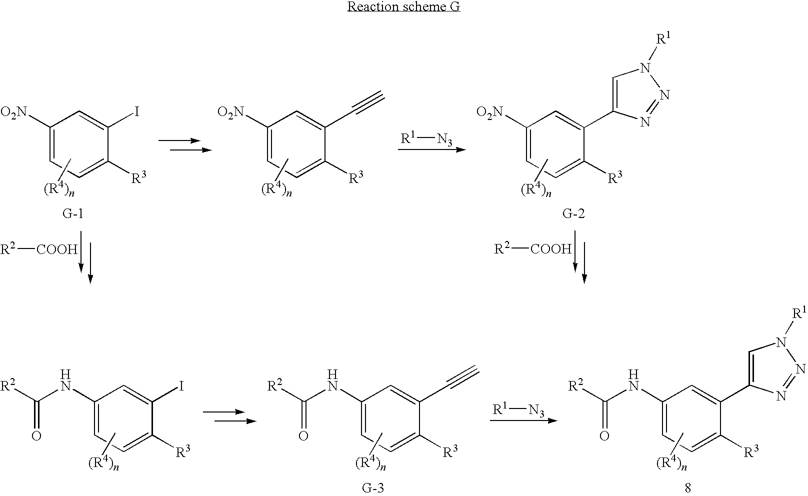

Compounds of type 8 are synthesised starting from G-1 either via the intermediates G-2 or G-3 by using Sonogashira coupling reactions, amide couplings and cycloadditions in the appropriate order, as described for compounds of type 1. The inverted amides of the compounds of type 2-7 are also prepared by similar transformations to those described in reaction scheme G.

### Synthesis of Examples 1a-1a1

### Method for Synthesising A-2a

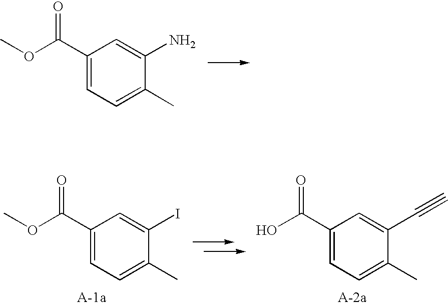

Methyl 3-amino-4-methylbenzoate (1.652 g, 10 mmol) is dissolved in 35% sulphuric acid (18 mL) and acetic acid (6 mL) and cooled to 0° C. Then a solution of sodium nitrite (0.76 g, 11 mmol) in 3 mL water is added dropwise, the mixture is stirred for 1 h at 0° C. and for 1 h at RT, then a solution of potassium iodide (2.0 g, 12 mmol) in 4 mL water is added and the mixture is stirred for 2 h. For working up the reaction mixture is combined with DCM, extracted twice, the combined organic phases are dried on sodium sulphate, filtered and evaporated down under reduced pressure. Chromatographic purification of the residue obtained through silica gel (5 EE in cyclohexane) yields A-1a (HPLC-MS: t_{Ret.}=3.89 min; MS (M+H)⁺=277).

Under protective gas A-1a (0.2 g, 0.724 mmol) together with bis-triphenylphosphine-palladium dichloride (25.4 mg, 0.036 mmol) and copper(I) iodide is added to abs. THF (3 mL) and triethylamine (1 mL). Trimethylsilyl-ethyne is added at RT to this reaction mixture and stirred overnight. For working up it is diluted with EE, poured onto 0.5 M ammonia solution and the aqueous phase is again extracted with EE. The combined organic phases are washed with 0.5 M hydrochloric acid and saturated NaCl solution, extracted again with EE, dried on sodium sulphate, filtered and evaporated down under reduced pressure.

The residue is combined with methanolic KOH (1 mL) and stirred for 2 h at RT. The reaction mixture is diluted with EE, poured onto 5% NaHCO₃ solution and extracted twice with EE. The combined organic phases are washed with saturated sodium chloride solution, dried on sodium sulphate, filtered and evaporated down under reduced pressure. Chromatographic purification through a short silica gel frit yields A-2a (HPLC-MS: t_{Ret.}=3.65 min; MS (M+H)⁺=175).

Analogously to this process other compounds A-2 may be obtained from the corresponding 3-aminobenzoic acid derivatives.

### Method for Synthesising the Intermediates B-1a

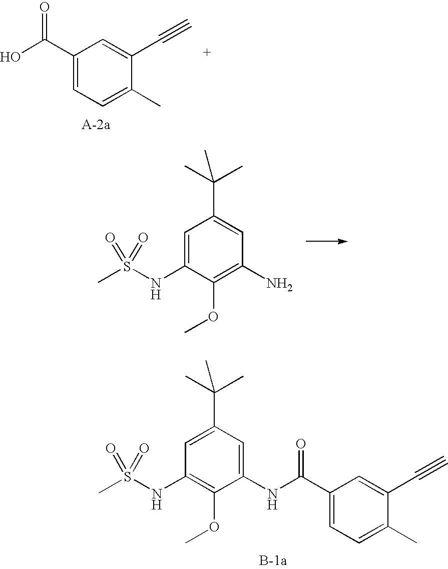

The carboxylic acid A-2a (647 mg, 4.04 mmol) is dissolved in abs. THF (10 mL) and abs. DCM (40 mL) and at RT combined dropwide with a-chloro-enamine reagent (Ghosez reagent, 0.577 mL, 4.41 mmol). After 1 h at RT the amine (1.00 g, 3.67 mmol) is added, DIPEA (1.973 mL, 11 mmol) is added dropwise and the mixture is stirred for 24 h. For working up it is diluted with EE, acidified with 1M hydrochloric acid solution, the aqueous phase is extracted several times with EE, the combined organic phases are dried on magnesium sulphate, filtered and evaporated down under reduced pressure. The residue remaining is purified by RP-HPLC separation and the corresponding compound B-1a is obtained (HPLC-MS: t_{Ret.}=2.25 min; MS (M+H)⁺=415).

Analogously to this process compounds B-1 in general are obtained from the corresponding A-2-intermediates.

### Method for Synthesising the Intermediates B-2a

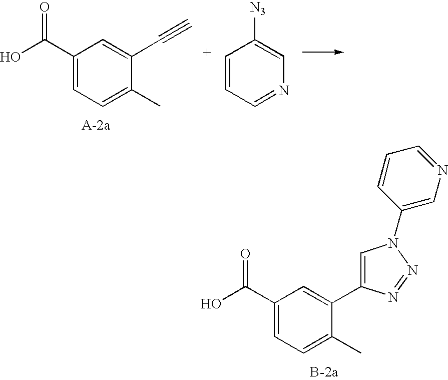

15 mL 1M sodium ascorbate solution and 20 mL 0.1 M CuSO₄ solution are added successively to the carboxylic acid A-2a (2.00 g, 12.5 mmol) and the azide (1.12 g, 9.32 mmol) dissolved in MeOH (200 mL). The mixture is stirred for 8 d at RT. The precipitate of B-2a formed is filtered off, washed with a little water and dried in vacuo (HPLC-MS: t_{Ret.}=1.64 min; MS (M+H)⁺=281).

Analogously to this process other compounds B-2 are obtained from the corresponding A-2-intermediates.

### Method for Synthesising Example 1a

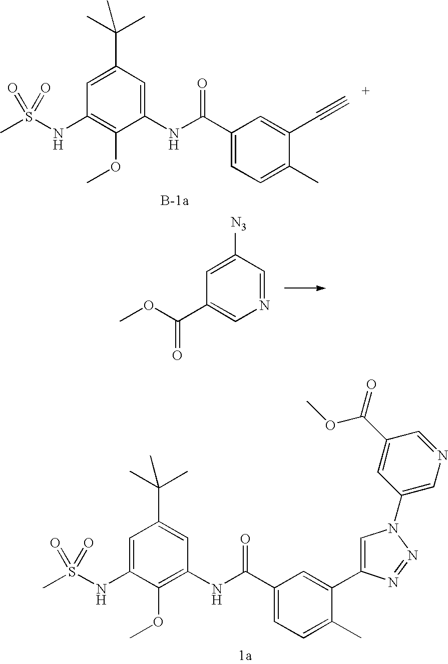

The alkyne B-1a (30 mg, 0.07 mmol) and the azide (16 mg, 0.09 mmol) are dissolved in 0.6 mL acetonitrile/MeOH (1:1), combined with 80 μL triethylamine and 145 μL 1M sodium ascorbate solution and after 1 min 140 μL 0.1M CuSO₄ solution are added. The reaction mixture is stirred overnight at RT, after the reaction is complete it is diluted with some DMF and filtered. Chromatographic purification by RP-HPLC yields 1a.

Analogously to this process Examples 1b-1z and other comparable compounds of type I are obtained from the corresponding intermediates B-1 (see the following Table).

### Method for Synthesising Example 1aa

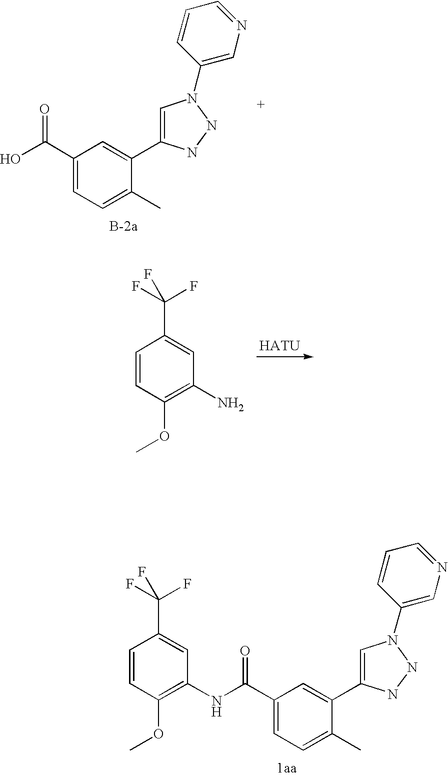

HATU (304 mg, 0.94 mmol) and Hünig base (152 μL, 0.91 mmol) are added at RT to a solution of carboxylic acid B-2a (154 mg, 0.55 mmol) in THF and the mixture is stirred for 30 min. Then the amine (125 mg, 0.66 mmol) is added and the mixture is stirred for 5 d at 50° C. The mixture is evaporated down in vacuo and dissolved in a little DMF. Chromatographic purification by RP-HPLC yields 1aa.

Analogously to this process Examples 1ab-1a1 and other comparable compounds of type 1 are obtained from the corresponding intermediates B-2 (see the following Table).

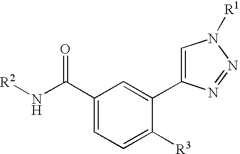

1a-1al

| | | | | t_{Ret} | |
|---|---|---|---|---|---|
| | | | | (HPLC) | MS |
| # | R¹ | R² | R³ | [min] | (M + H)⁺ |
| 1a | 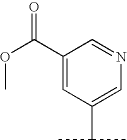 | 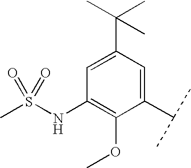 | CH₃ | 2.18 | 593 |
| 1b | 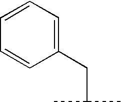 | 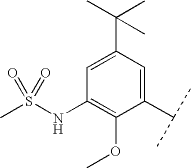 | CH₃ | 2.26 | 548 |
| 1c | 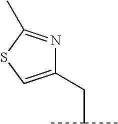 | 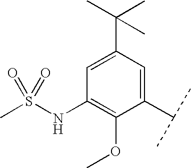 | CH₃ | 2.12 | 569 |
| 1d | 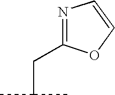 | 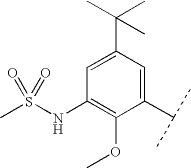 | CH₃ | 2.00 | 539 |
| 1e | 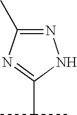 | 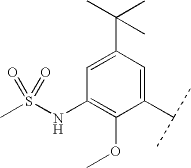 | CH₃ | 1.98 | 539 |
| 1f | 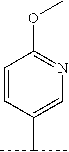 | 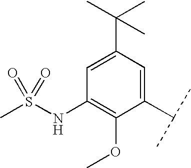 | CH₃ | | |
| 1g | 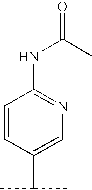 | 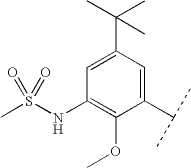 | CH₃ | | |
| 1h | 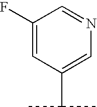 | 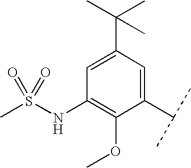 | CH₃ | | |
| 1i | 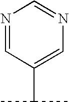 | 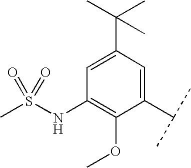 | CH₃ | | |
| 1j | 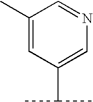 | 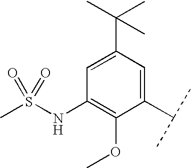 | CH₃ | | |
| 1k | 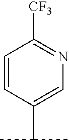 | 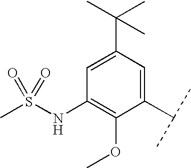 | CH₃ | | |
| 1l | 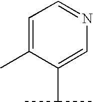 | 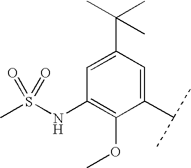 | CH₃ | | |
| 1m | 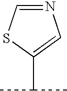 | 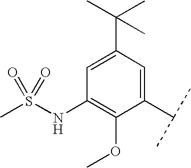 | CH₃ | | |
| 1n | 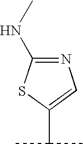 | 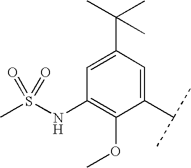 | CH₃ | | |
| 1o | 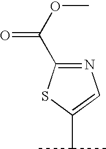 | 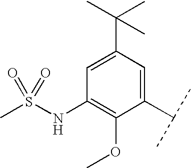 | CH₃ | | |
| 1p | 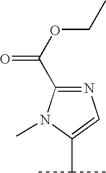 | 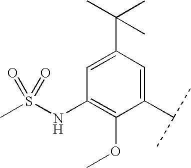 | CH₃ | | |
| 1q | 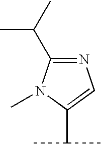 | 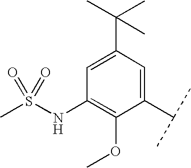 | CH₃ | | |
| 1r | 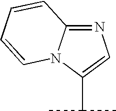 | 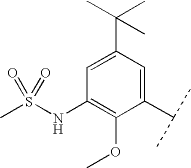 | CH₃ | | |
| 1s | 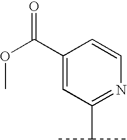 | 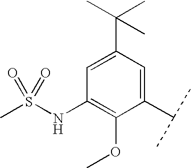 | CH₃ | | |
| 1t | 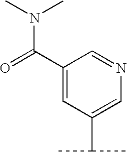 | 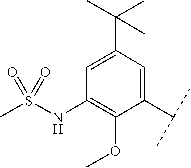 | CH₃ | | |
| 1u | 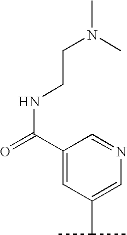 | 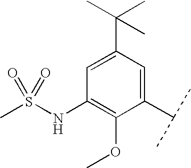 | CH₃ | | |
| 1v | 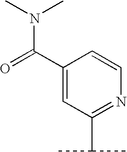 | 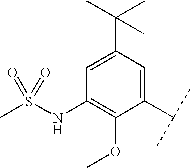 | CH₃ | | |
| 1w | 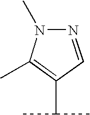 | 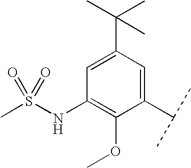 | CH₃ | | |
| 1x | 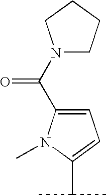 | 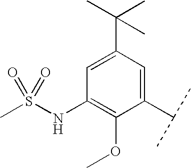 | CH₃ | | |
| 1y | 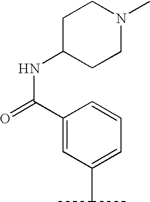 | 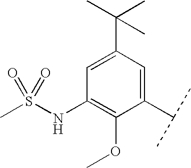 | CH₃ | | |
| 1z | 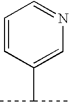 | 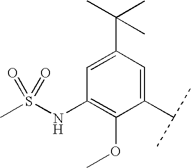 | Cl | | |
| 1aa | 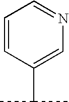 | 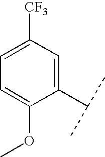 | CH₃ | 2.27 | 454 |
| 1ab | 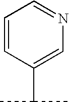 | 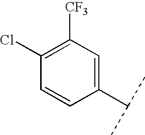 | CH₃ | 2.35 | 458 |
| 1ac | 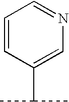 | 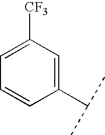 | CH₃ | 2.23 | 424 |
| 1ad | 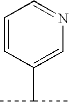 | 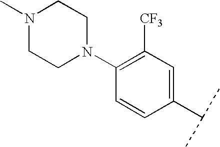 | CH₃ | | |
| 1ae | 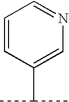 | 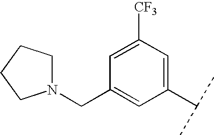 | CH₃ | | |
| 1af | 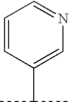 | 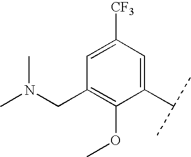 | CH₃ | | |
| 1ag | 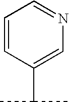 | 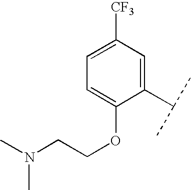 | CH₃ | | |
| 1ah | 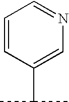 | 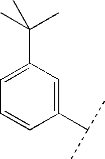 | CH₃ | | |
| 1ai | 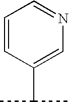 | 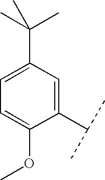 | CH₃ | | |
| 1aj | 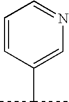 | 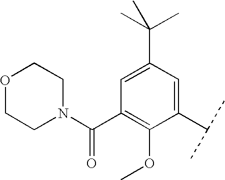 | CH₃ | | |
| 1ak | 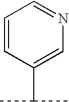 | 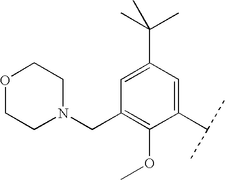 | CH₃ | | |
| 1al | 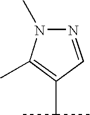 | 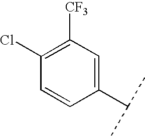 | CH₃ | | |

### Synthesis of Examples 2a-2w

### Method for Synthesising C-1a

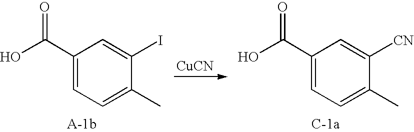

A-1b (1.00 g, 3.82 mmol) is placed in anhydrous DMF (4 mL), combined with CuCN in (449 mg, 4.96 mmol) and stirred for 20 h at 100° C. Aqueous working up and evaporation using the rotary evaporator yields C-1a (HPLC-MS: t_{Ret.}=1.39 min; MS (M+H)⁺=162). Analogously to this process are other compounds C-1 are obtained from the corresponding 3-iodobenzoic acids.

### Method for Synthesising C-2a

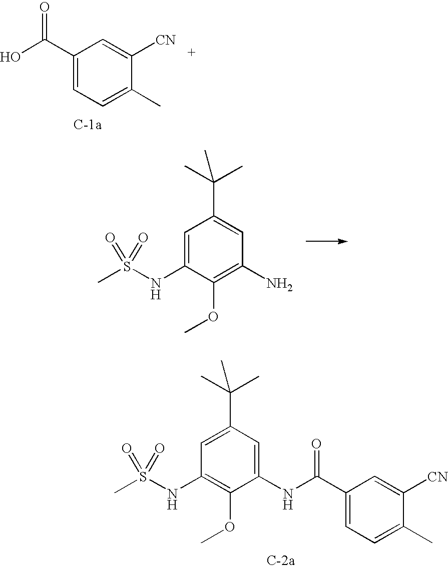

C-1a (200 mg, 1.24 mmol) in anhydrous THF/DCM (10 ml, 1:1) is mixed dropwise with oxalyl chloride (119 μL, 1.37 mmol) and one drop of DMF. The mixture is stirred for 2 h at RT and the mixture is then evaporated down completely using the rotary evaporator. The residue is taken up in DCM (1 mL) and combined with THF (1 mL). A solution of the amine (383 mg, 1.24 mmol) in THF and Hünig base (633 μL, 3.72 mmol) are added dropwise and the reaction mixture is stirred for 3 h at RT. Aqueous working up and recrystallisation from EtOH yields C-2a (HPLC-MS: t_{Ret.}=2.11 min; MS (M+H)⁺=416). Analogously to this process other compounds C-2 are generally obtained from the corresponding C-1-intermediates.

### Method for Synthesising C-3a

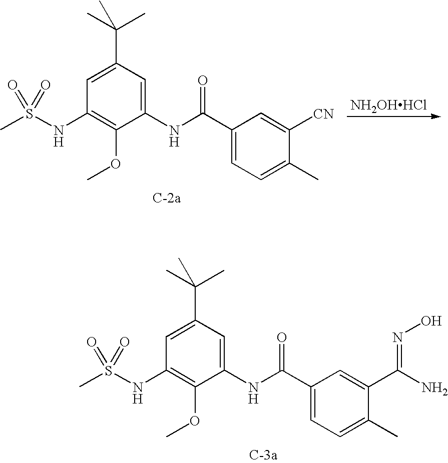

C-2a (100 mg, 0.24 mmol), hydroxylamine hydrochloride (36.8 mg, 0.53 mmol) and NEt₃ (84 μL, 0.60 mmol) are refluxed in EtOH (600 μL) for 2 h. Evaporation using the rotary evaporator and chromatographic purification by RP-HPLC yields C-3a (HPLC-MS: t_{Ret.}=0.18-1.36 min; MS (M+H)⁺=449).

Analogously to this process other compounds C-3 are generally obtained from the corresponding C-2-intermediates.

### Method for Synthesising Example 2a

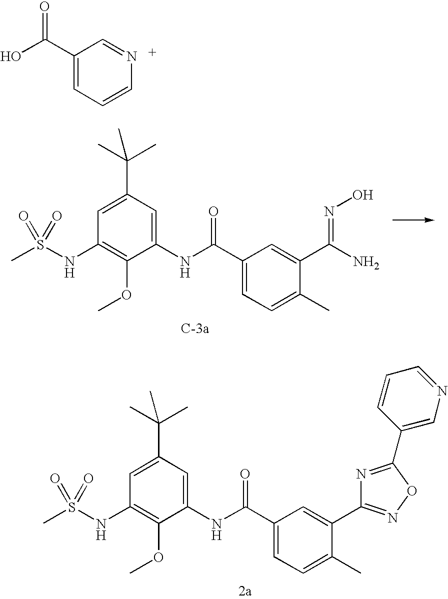

The carboxylic acid (13 mg, 0.11 mmol) is dissolved in DMF (500 μL), combined with Hünig base (80 μL, 0.44 mmol) and TBTU (34 mg, 0.11 mmol) and stirred at RT for 15 min. Then C-3a (40 mg, 0.09 mmol) is added and the mixture is stirred for 3 h at RT. The mixture is briefly heated to 100° C. After cooling and chromatographic purification by RP-HPLC, 2a is obtained (see the following Table).

Analogously to the processes described above Examples 2b-2w and other comparable compounds of type 2 may be synthesised using the corresponding amine R²—NH₂ and the corresponding carboxylic acid R¹—COOH.

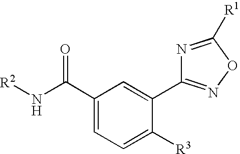

2a-2w

| | | | | t_{Ret} | |
|---|---|---|---|---|---|
| | | | | (HPLC) | MS |
| # | R¹ | R² | R³ | [min] | (M + H)⁺ |
| 2a | 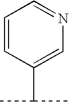 | 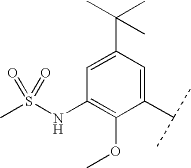 | CH₃ | 2.28 | 536 |
| 2b | 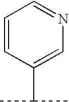 | 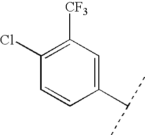 | CH₃ | 2.51 | 459 |
| 2c | 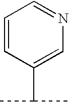 | 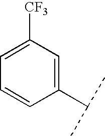 | CH₃ | | |
| 2d | 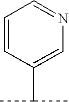 | 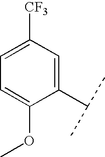 | CH₃ | | |
| 2e | 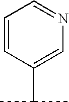 | 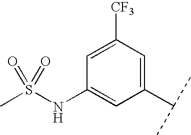 | CH₃ | | |
| 2f | 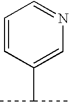 | 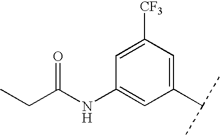 | CH₃ | | |
| 2g | 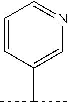 | 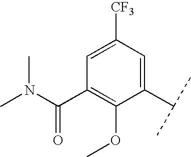 | CH₃ | | |
| 2h | 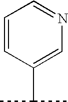 | 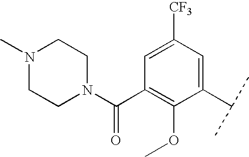 | CH₃ | | |
| 2i | 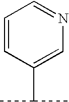 | 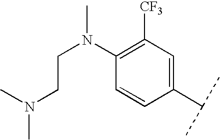 | CH₃ | | |
| 2j | 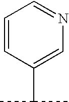 | 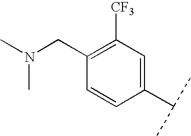 | CH₃ | | |
| 2k | 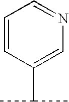 | 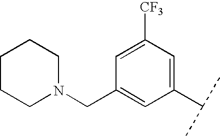 | CH₃ | | |
| 2l | 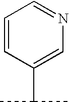 | 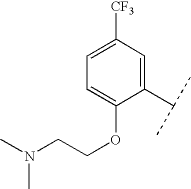 | CH₃ | | |
| 2m | 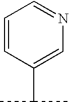 | 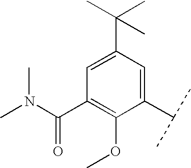 | CH₃ | | |
| 2n | 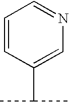 | 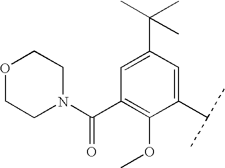 | CH₃ | | |
| 2o | 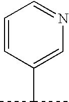 | 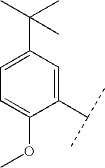 | CH₃ | | |
| 2p | 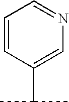 | 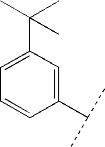 | CH₃ | | |
| 2q | 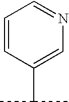 | 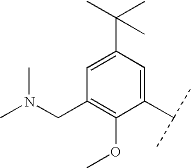 | CH₃ | | |
| 2r | 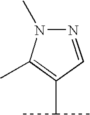 | 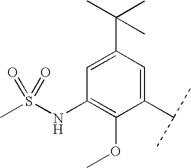 | CH₃ | | |
| 2s | 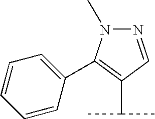 | 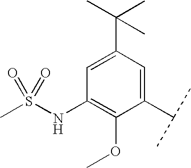 | CH₃ | | |
| 2t | 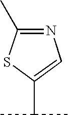 | 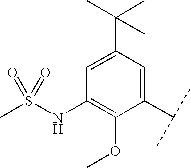 | CH₃ | | |
| 2u | 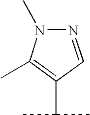 | 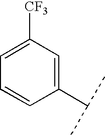 | Cl | | |
| 2v | 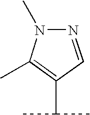 | 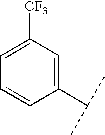 | OMe | | |
| 2w | 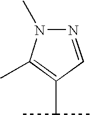 | 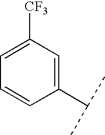 | CF₃ | | |

### Synthesis of Examples 3a-3w

### Method for Synthesising C-4a

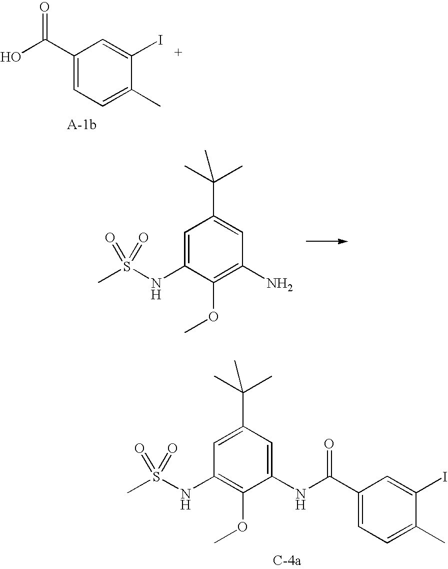

Benzoic acid A-1b (5.08 g, 19.4 mmol) is dissolved in 150 mL anhydrous DCM/THF (2:1) and combined dropwise with oxalyl chloride (1.76 mL, 20.2 mmol). Then a few drops of DMF are added and the mixture is stirred for 2 h at RT. The mixture is evaporated down completely using the rotary evaporator, dissolved in 50 mL DCM and a solution of the amine (5.70 g, 18.5 mmol) and Hünig base in THF (9.4 mL, 55.1 mmol) is added dropwise. Then the mixture is stirred for 3 h at RT. After aqueous working up and recrystallisation from EtOH, C-4a is obtained.

Analogously to this process other compounds C-4 are agenerally obtained from the corresponding 3-iodobenzoic acids and the corresponding amines R²—NH₂.

### Method for Synthesising C-5a

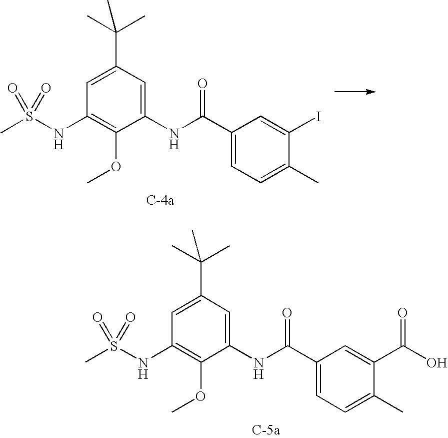

C-4-a (1.00 g, 1.94 mmol) is dissolved in anhydrous THF (40 mL) under protective gas, cooled to −20° C. and combined with iPrMgCl solution (4.31 mL, 1.8M). The mixture is stirred at this temperature for 2 h. Then CO₂ is piped through the reaction mixture. After 1 h, NH₄Cl solution is added and the mixture is extracted twice with EE. The combined organic phases are extracted three times with 2M NaOH solution, the combined aqueous phases are then acidified with 6M HCl and extracted several times with EE. Drying through Na₂SO₄ and evaporation using the rotary evaporator yields C-5a.

Analogously to this process other compounds C-5 are generally obtained from the corresponding compounds C-4.

### Method for Synthesising Example 3a

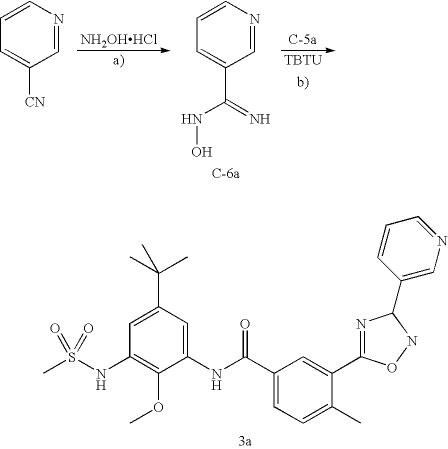

a) 3-cyanopyridine (500 mg, 4.80 mmol), hydroxylamine hydrochloride (734 mg, 10.6 mmol) and NEt₃ (1.5 mL, 11.0 mmol) are refluxed in MeOH (10 mL) for 2 h. Then the reaction mixture is evaporated down using the rotary evaporator and worked up in aqueous form. The hydroxylamidine C-6a (HPLC-MS: t_{Ret.}=0.00 min; MS (M+H)⁺=138) is obtained.

Analogously to this process generally, hydroxylamidines C-6 are obtained from the corresponding nitriles.

b) C-5a (30 mg, 0.07 mmol) is dissolved in DMF (300 μL), combined with Hünig base (62 μL, 0.35 mmol) and TBTU (22.2 mg, 0.07 mmol) and stirred for 15 min at RT. Then C-6a (11.4 mg, 0.08 mmol) is added thereto and the mixture is stirred for 4 d at RT. After aqueous working up and evaporation using the rotary evaporator the residue is taken up in DMF (1 mL) and stirred for 4 h at 110° C. After cooling 3a is obtained by durch chromatographic purification by RP-HPLC.

Analogously to the processes described above Examples 3b-3w and other comparable compounds of type 3 are synthesised using the corresponding amine R²—NH₂ and the corresponding nitrile R¹—CN.

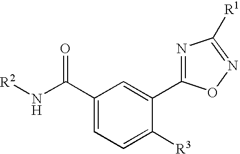

3a-3w

| | | | | t_{Ret} | |
|---|---|---|---|---|---|
| | | | | (HPLC) | MS |
| # | R¹ | R² | R³ | [min] | (M + H)⁺ |
| 3a | 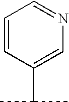 | 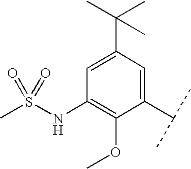 | CH₃ | 2.29 | 536 |
| 3b | 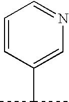 | 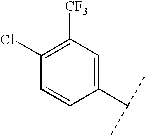 | CH₃ | | |
| 3c | 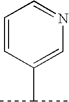 | 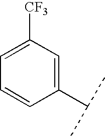 | CH₃ | | |
| 3d | 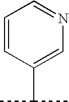 | 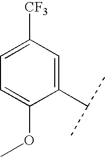 | CH₃ | | |
| 3e | 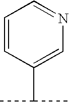 | 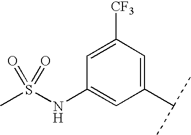 | CH₃ | | |
| 3f | 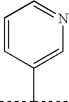 | 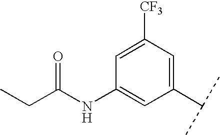 | CH₃ | | |
| 3g | 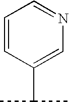 | 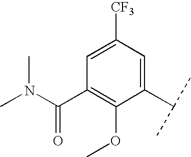 | CH₃ | | |
| 3h | 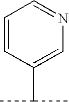 | 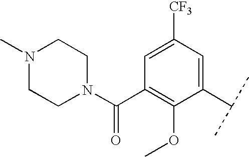 | CH₃ | | |
| 3i | 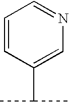 | 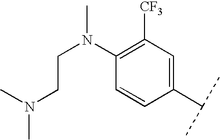 | CH₃ | | |
| 3j | 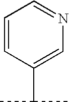 | 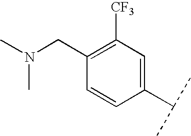 | CH₃ | | |
| 3k | 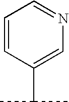 | 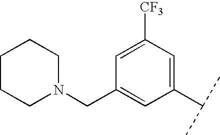 | CH₃ | | |
| 3l | 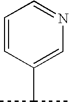 | 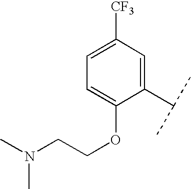 | CH₃ | | |
| 3m | 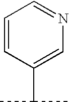 | 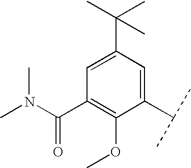 | CH₃ | | |
| 3n | 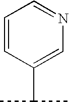 | 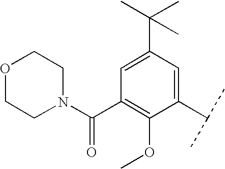 | CH₃ | | |
| 3o | 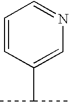 | 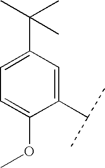 | CH₃ | | |
| 3p | 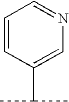 | 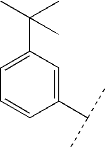 | CH₃ | | |
| 3q | 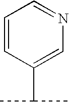 | 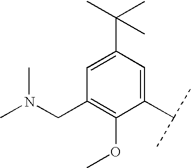 | CH₃ | | |
| 3r | 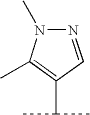 | 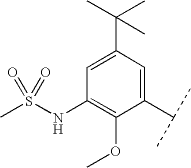 | CH₃ | | |
| 3s | 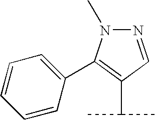 | 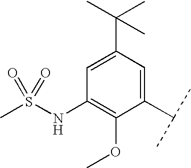 | CH₃ | | |
| 3t | 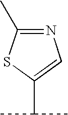 | 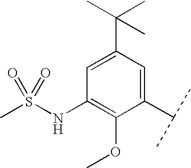 | CH₃ | | |
| 3u | 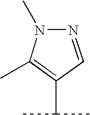 | 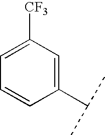 | Cl | | |
| 3v | 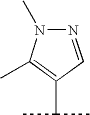 | 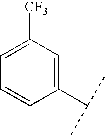 | OMe | | |
| 3w | 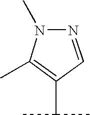 | 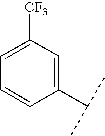 | CF₃ | | |

### Synthesis of Examples 4a-a, 4b-a, 5a-a and 5b-a-5b-h

Compounds of type 4a, 4b, 5a and 5b may be synthesised analogously to the routes mentioned in Reaction Scheme D.

### Method for Synthesising D-2a

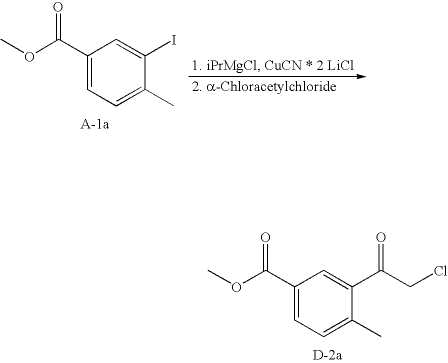

Ester A-1a (5.00 g, 18.1 mmol) is taken up under protective gas in anhydrous THF (77 mL), cooled to −30° C. and combined with iPrMgCl (13.3 mL, 1.5 M in THF) with stirring. After 1.5 h, CuCN.2 LiCl (19.9 mL, 1.0 M in THF) is added and the mixture is stirred for 1 h at −30° C. before a-chloroacetyl chloride (476 μL, 19.9 mmol) is added and allowed to thaw at RT. After aqueous working up and chromatography (NP, cyclohexane/EE) D-2a is obtained.

### Method for Synthesising D-3a

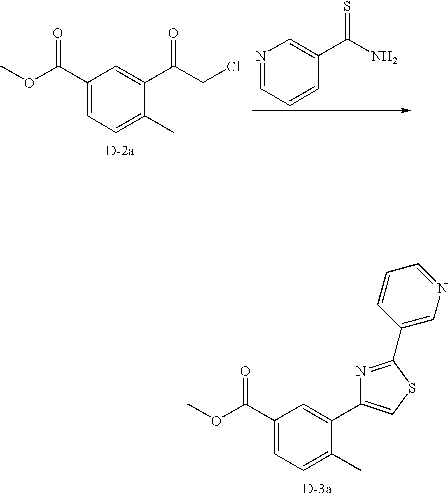

D-2a (180 mg, 0.79 mmol) and thionicotinamide (110 mg, 0.79 mmol) are placed in MeOH (1.6 mL) and heated to 100° C. for 20 min in the microwave reactor. After aqueous working up and chromatographic purification by RP-HPLC, D-3a is obtained (HPLC-MS: t_{Ret.}=2.09 min; MS (M+H)⁺=311).

### Method for Synthesising Example 5b-a

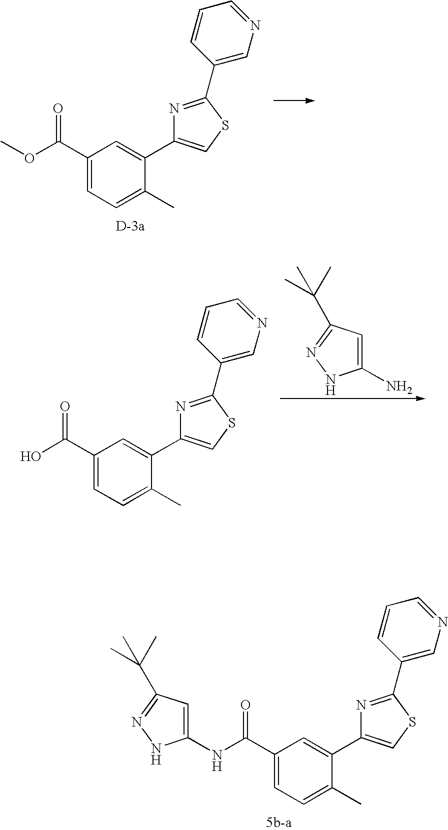

D-3a (89.0 mg, 0.29 mmol) is taken up in 1,4-dioxane (5 mL) and H₂O (5 mL), combined with LiOH (20.6 mg, 0.86 mmol) and stirred for 3 h at RT. After aqueous working up the free carboxylic acid is obtained (HPLC-MS: t_{Ret.}=1.24 min; MS (M+H)⁺=297), which is used without further purification.

The carboxylic acid obtained (35.0 mg, 0.12 mmol) is taken up in THF (0.6 mL) and DCM (0.6 mL), combined with oxalyl chloride (20 μL, 0.24 mmol) and one drop of DMF and stirred for 30 min at RT. The reaction solution is evaporated down completely at the rotary evaporator, taken up in THF (0.6 mL) and DCM (0.6 mL) again, combined with NEt₃ (38 μt, 0.27 mmol) and 5-amino-3-tert-butylpyrazol (16.4 mg, 0.12 mmol) and stirred overnight at RT. 5b-a is obtained by chromatographic purification by RP-HPLC.

Analogously to the processes described above Examples 5b-b-5b-h and other comparable compounds of type 5b are synthesised using the corresponding amine R²—NH₂ and the corresponding carboxylic acid ester D-3.

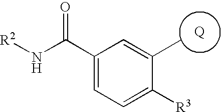

4a-a, 4b-a, 5a-a and 5b-a-5b-h

| # | R¹ | R² | R³ | 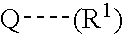 | t_{Ret} (HPLC) [min] | MS (M + H)⁺ |
|---|---|---|---|---|---|---|
| 4a-a | 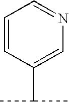 | 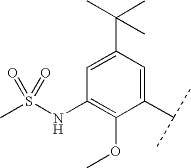 | CH₃ | 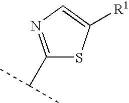 | | |
| 4b-a | 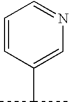 | 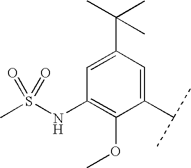 | CH₃ | 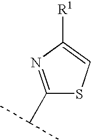 | | |
| 5a-a | 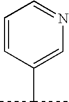 | 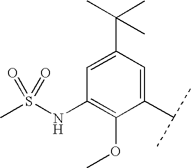 | CH₃ | 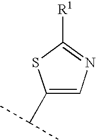 | | |
| 5b-a | 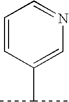 | 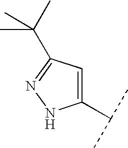 | CH₃ | 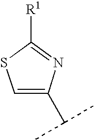 | 2.08 | 418 |
| 5b-b | 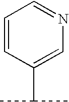 | 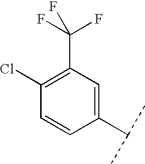 | CH₃ | 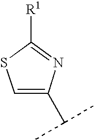 | 2.54 | 474 |
| 5b-c | 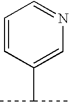 | 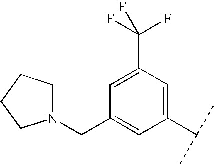 | CH₃ | 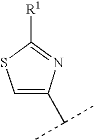 | | |
| 5b-d | 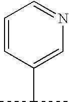 | 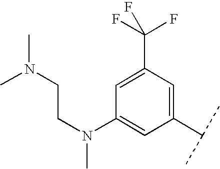 | CH₃ | 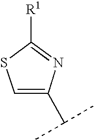 | | |
| 5b-e | 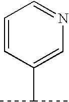 | 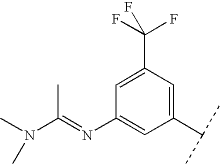 | CH₃ | 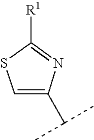 | | |
| 5b-f | 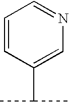 | 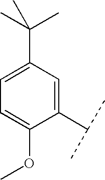 | CH₃ | 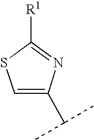 | | |
| 5b-g | 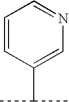 | 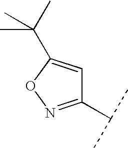 | CH₃ | 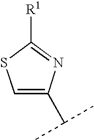 | | |
| 5b-h | 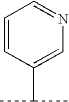 | 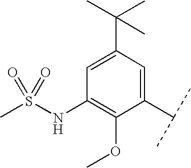 | CH₃ | 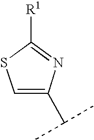 | | |

### Synthesis of Examples 6a-61

### Method for Synthesising E-3a

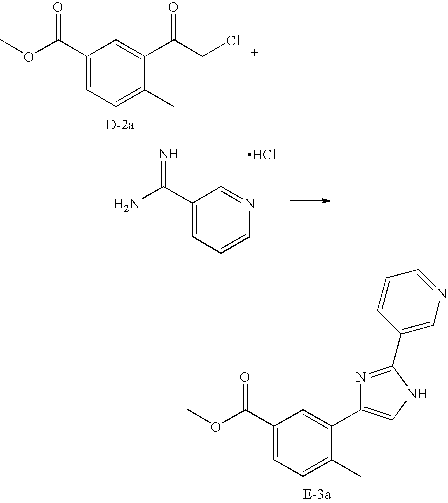

D-2a (1.00 g, 4.41 mmol), 3-amidinopyridine hydrochloride (695 mg, 4.41 mmol) and K₂CO₃ (1.22 g, 8.82 mmol) are taken up in MeOH (9 mL) and heated for 15 min in the microwave reactor to 100° C. After elimination of the solvent using the rotary evaporator the residue is combined with MeCN. The precipitate formed is filtered off and the filtrate is evaporated down again. The product thus obtained E-3a is used for the next step without any further purification (HPLC-MS: t_{Ret.}=1.74 min; MS (M+H)⁺=294).

### Method for Synthesising Example 6a

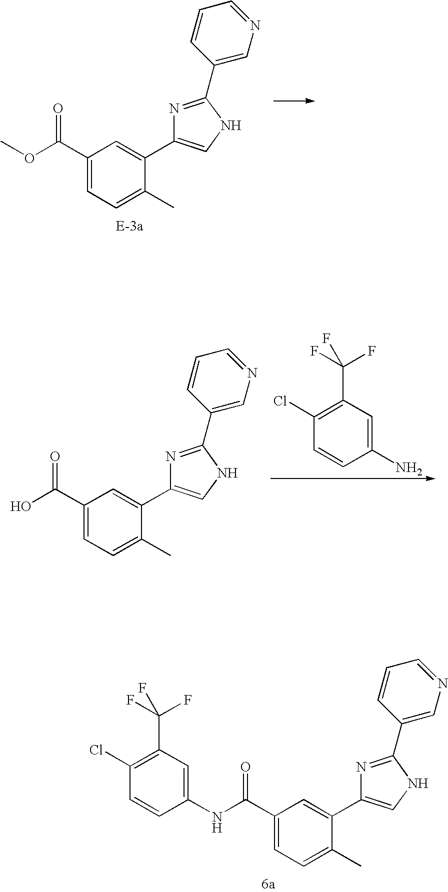

E-3a (1.29 g, 4.40 mmol) is taken up in 1,4-dioxane (10 mL) and H₂O (10 mL), combined with LiOH (316 mg, 13.2 mmol) and stirred for 1 h at RT. Then the volatile constituents are eliminated using the rotary evaporator. The residue is taken up in H₂O and adjusted to pH 2 with hydrochloric acid. The precipitate thus formed is filtered off and discarded. The filtrate is adjusted to pH 5 with sodium hydroxide solution, whereupon the carboxylic acid is precipitated (HPLC-MS: t_{Ret.}=0.23 min; MS (M+H)⁺=280).

The free carboxylic acid (70.0 mg, 0.25 mmol) is placed in DMF (1 mL) and NEt₃ (87 μL, 0.63 mmol). Then HATU (238 mg, 0.63 mmol) and 4-chloro-3-(trifluoromethyl)aniline (73.5 mg, 0.38 mmol) are added and the mixture is left overnight at RT with stirring. 6a is obtained by RP-HPLC.

Analogously to the processes described above Examples 6b-6i and other comparable compounds of type 6 are synthesised using the corresponding amine R²—NH₂ and the corresponding esters E-3.

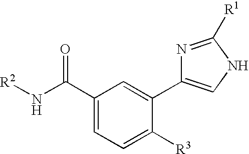

6a-6i

| | | | | t_{Ret} | |
|---|---|---|---|---|---|
| | | | | (HPLC) | MS |
| # | R¹ | R² | R³ | [min] | (M + H)⁺ |
| 6a | 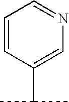 | 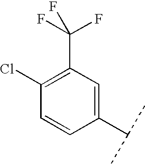 | CH₃ | 2.25 | 457 |
| 6b | 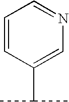 | 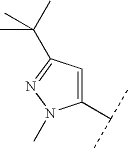 | CH₃ | 1.87 | 415 |
| 6c | 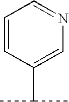 | 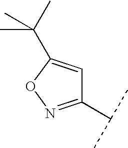 | CH₃ | 2.04 | 402 |
| 6d | 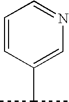 | 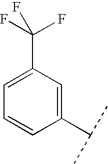 | CH₃ | | |
| 6e | 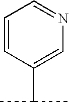 | 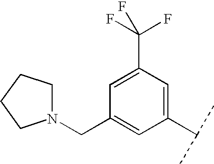 | CH₃ | | |
| 6f | 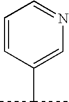 | 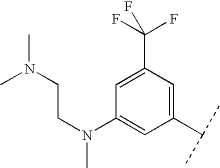 | CH₃ | | |
| 6g | 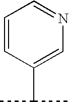 | 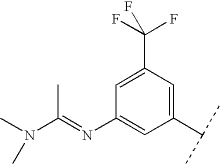 | CH₃ | | |
| 6h | 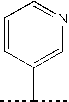 | 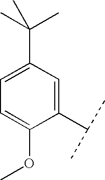 | CH₃ | | |
| 6i | 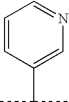 | 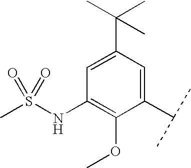 | CH₃ | | |

### Synthesis of Examples 7a-7j

### Method for Synthesising B-1b

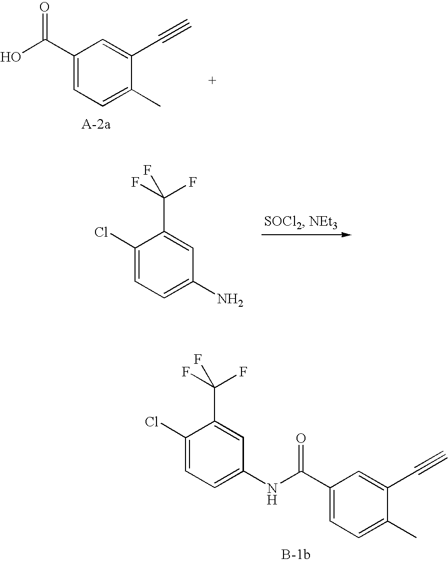

Carboxylic acid A-2a (250 mg, 1.56 mmol) is taken up in MeCN (2.4 mL), thionyl chloride (1.2 mL, 15.6 mmol) is added while cooling with ice and the mixture is heated to 80° C. for 2 min in the microwave reactor. Then it is evaporated down using the rotary evaporator and the residue is taken up in MeCN (3.1 mL). NEt₃ (216 μL, 1.56 mmol) and 4-chloro-3-(trifluoromethyl)aniline (305 mg, 1.56 mmol) are added successively and the mixture is stirred for 10 min at RT. After aqueous working up B-1b (HPLC-MS: t_{Ret.}=2.48 min; MS (M+H)⁺=338) is obtained, which is used without further purification.

### Method for Synthesising Example 7a

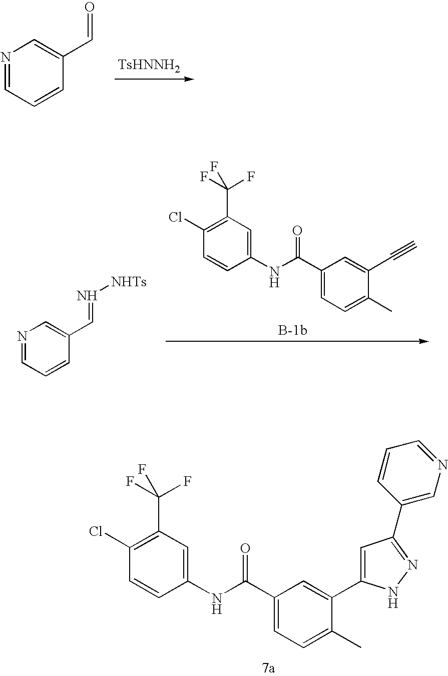

Pyridine-3-carbaldehyde (56 μL, 0.59 mmol) is placed in EtOH (590 μL), combined with p-TsHNNH2 (110 mg, 0.59 mmol) and heated for 2 min in the microwave reactor to 80° C. Then the solvent is distilled off using the rotary evaporator and the residue (tosylhydrazone) is taken up in MeCN (1 mL). Sodium hydroxide solution (74 μL, 8 M) and H₂O (50 μL) is added and the mixture is stirred for 30 min at RT before the alkyne B-1b (200 mg, 0.59 mmol) is added. The reaction mixture is heated for 15 min at 100° C. in the microwave reactor. 7a is obtained by chromatographic purification by RP-HPLC.

Analogously to the processes described above Examples 7b-7j and other comparable compounds of type 7 are synthesised using the corresponding aldehydes and the alkynes B-1.

7a-7j

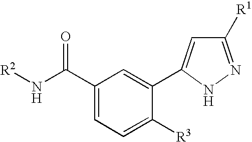

| | | | | t_{Ret} | |
|---|---|---|---|---|---|
| | | | | (HPLC) | |
| # | R¹ | R² | R³ | [min] | MS (M + H)⁺ |
| 7a | 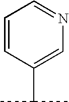 | 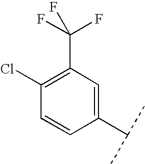 | CH₃ | 2.25 | 457 |
| 7b | 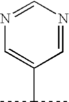 | 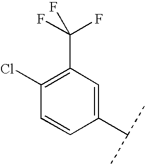 | CH₃ | 2.19 | 458 |
| 7c | 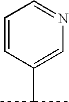 | 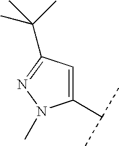 | CH₃ | 1.92 | 415 |
| 7d | 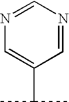 | 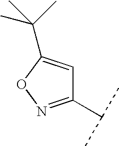 | CH₃ | 2.01 | 403 |
| 7e | 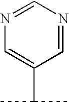 | 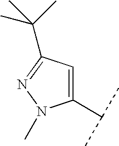 | CH₃ | 1.84 | 416 |
| 7f | 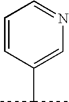 | 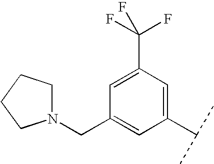 | CH₃ | | |
| 7g | 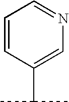 | 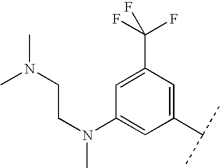 | CH₃ | | |
| 7h | 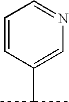 | 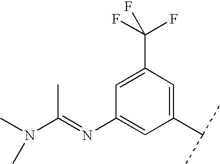 | CH₃ | | |
| 7i | 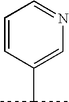 | 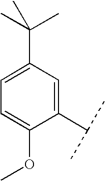 | CH₃ | | |
| 7j | 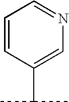 | 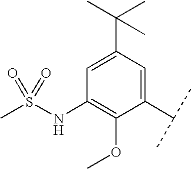 | CH₃ | | |

### Synthesis of Examples 8a-8p

The following Examples 8a-8p may be synthesised analogously to the route described in Reaction Scheme G.

8a-8p

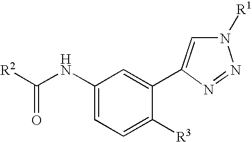

| | | | | t_{Ret} | |
|---|---|---|---|---|---|
| | | | | (HPLC) | MS |
| # | R¹ | R² | R³ | [min] | (M + H)⁺ |
| 8a | 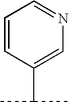 | 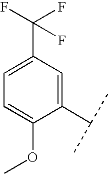 | CH₃ | | |
| 8b | 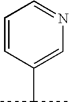 | 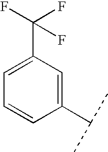 | CH₃ | | |
| 8c | 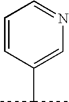 | 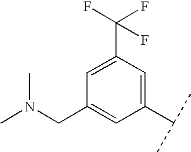 | CH₃ | | |
| 8d | 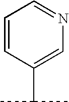 | 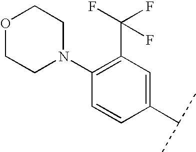 | CH₃ | | |
| 8e | 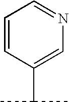 | 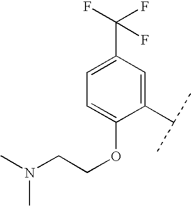 | CH₃ | | |
| 8f | 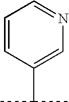 | 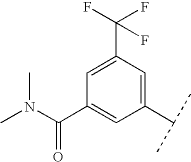 | CH₃ | | |
| 8g | 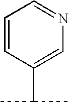 | 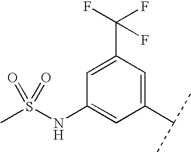 | CH₃ | | |
| 8h | 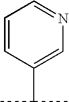 | 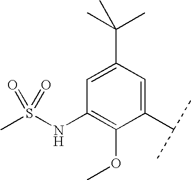 | CH₃ | | |
| 8i | 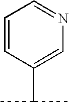 | 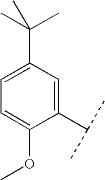 | CH₃ | | |
| 8j | 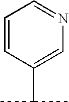 | 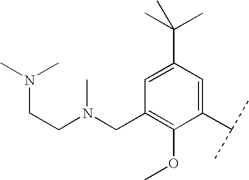 | CH₃ | | |
| 8k | 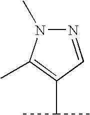 | 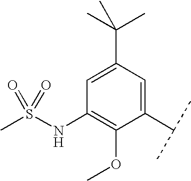 | CH₃ | | |
| 8l | 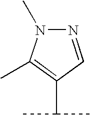 | 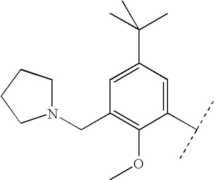 | CH₃ | | |
| 8m | 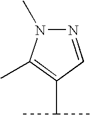 | 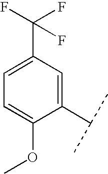 | CH₃ | | |
| 8n | 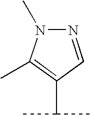 | 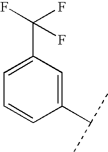 | CH₃ | | |
| 8o | 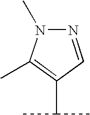 | 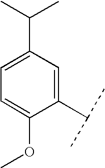 | CH₃ | | |
| 8p | 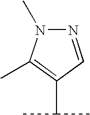 | 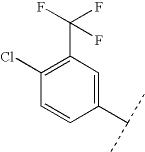 | CH₃ | | |

The following Examples describe the biological activity of the compounds according to the invention without restricting the invention to these Examples.

Compounds of general formula (1) are characterised by their wide range of applications in the therapeutic field. Particular mention should be made of those applications in which the inhibition of specific signal enzymes, particularly the inhibiting effect on the proliferation of cultivated human tumour cells but also the proliferation of other cells, such as endothelial cells, for example, plays a part.

Kinase Test B-Raf (V600E)

In a dilution series, 10 μL aliquots of test substance solution are placed in a multiwell plate. The dilution series is selected so as to cover a range of concentrations from 2 μM to 0.128 or 0.017 nM. If necessary the initial concentration is changed from 2 μM to 10 or 0.4 μM and further dilution is carried out accordingly. The final concentration of DMSO is 5%. 10 μL of the B-Raf (V600E) kinase solution are pipetted in (containing 2.5 ng B-Raf (V600E)-kinase in 20 mM TrisHCl pH 7.5, 0.1 mM EDTA, 0.1 mM EGTA, 0.286 mM sodium orthovanadate, 10% glycerol, 1 mg/mL bovine serum albumin, 1 mM dithiothreitol) and incubated for 1 h at RT with agitation. The kinase reaction is started by the addition of 20 μL ATP solution [final concentration: 250 μM ATP, 30 mM Tris-HCl pH 7.5, 0.02% Brij, 0.2 mM sodium-orthovanadate, 10 mM magnesium acetate, phosphatase cocktail (Sigma, #P2850, dilution recommended by the manufacturer), 0.1 mM EGTA] and 10 μL MEK1 solution [containing 50 ng biotinylated MEK1 (prepared from purified MEK1 according to standard procedure, e.g. with reagent EZ-Link Sulfo-NHS-LC-Biotin, Pierce, #21335) in 50 mM Hepes pH 7.5, 150 mM NaCl, 10% glycerol, 0.02% Brij-35, 0.2 mM PMSF, 0.2 mM benzamidine] and carried out for 60 min at RT with constant agitation. The reaction is stopped by the addition of 12 μL of a 100 mM EDTA solution and incubated for a further 5 min. 55 μL of the reaction solution are transferred into a streptavidine-coated plate (e.g. Streptawell HighBond, Roche, #11989685001) and shaken gently for 1 h at RT, in order to bind biotinylated MEK1 to the plate. After removal of the liquid the plate is washed five times with 200 μL of 1×PBS, and 100 μL solution of primary antibody plus europium-labelled secondary antibody [Anti Phospho-MEK (Ser217/221), Cell Signaling #9121 and Eu-N1 labeled goat-anti-rabbit antibody, Perkin Elmer #AD01015], the primary antibody is diluted 1:2000 and the secondary antibody is added to 0.4-0.5 μg/mL diluted in Delfia Assay Buffer (Perkin Elmer, #1244-111). After 1 h agitation at RT the solution is poured away and washed five times with 200 μL Delfia Wash Buffer (Perkin Elmer #4010-00101244-114). After the addition of 200 μL Enhancement Solution (Perkin Elmer #4001-00101244-105) the preparation is shaken for 10 min at RT and then measured in a Wallac Victor using the programme “Delfia Time Resolved Fluorescence (Europium)”.

IC₅₀ values are determined from these dosage-activity curves using the software programme (GraphPadPrizm).

The example compounds 1a to 1e, 1aa to 1ac, 2a, 2b, 3a, 5b-a, 5b-b, 6a to 6c and 7a to 7e exhibit a good to very good inhibitory effect in this B-Raf (V600E) inhibition test, i.e. They have an IC₅₀ value of less than 0.5 μM, generally less than 200 nM.

Measurement of the Inhibition of Proliferation on Cultivated Human Melanoma Cells (SK-MEL28)

To measure proliferation on cultivated human tumour cells, cells of melanoma cell line SK-MEL28 [American Type Culture Collection (ATCC)] are cultivated in MEM medium, supplemented with 10% foetal calf serum, 2% sodium bicarbonate, 1 mM sodium pyruvate, 1% non-essential amino acids (e.g. from Cambrex, #BE13-114E) and 2 mM glutamine. SK-MEL28 cells are placed in 96-well flat-bottomed plates at a density of 2500 cells per well in supplemented MEM medium (see above) and incubated overnight in an incubator (at 37° C. and 5% CO₂). The active substances are added to the cells in various concentrations so as to cover a range of concentrations from 50 μM to 3.2 nM. If necessary the initial concentration is changed from 50 μM to 10 μM or 2 μM and further dilution (to 0.6 nM or 0.12 nM) is carried out accordingly. After a further 72 hours incubation, 20 μl AlamarBlue reagent (Serotec Ltd., #BUF012B) is added to each well, and the cells are incubated for a further 3-6 hours. The colour change of the AlamarBlue reagent is determined in a fluorescence spectrophotometer (e.g. Gemini, Molecular Devices). EC₅₀ values are calculated using the software programme (GraphPadPrizm).

The example compounds 1a, 1aa to 1ac, 3a, 5b-a, 6c and 7a to 7e exhibit a good to very good activity in the cellular SK-MEL28 assay, i.e. They have an EC₅₀ value of less than 5 μM.

Measurement of the Inhibition of Proliferation on Cultivated Human Melanoma Cells (A375)

To measure proliferation on cultivated human tumour cells, cells of melanoma cell line A375 [American Type Culture Collection (ATCC)] are cultivated in MEM medium, supplemented with 10% foetal calf serum and 2% sodium bicarbonate. Test substances are tested on A375 cells according to the method described for SK-MEL28 cells (see above).

The example compounds 1a, 1aa to 1ac, 2a, 2b, 3a, 5b-a, 6c and 7a to 7e exhibit a good to very good activity in the cellular A375 assay, i.e. They have an EC₅₀ value of less than 5 μM.

The substances of the present invention are B-Raf kinase inhibitors. As can be demonstrated by DNA staining followed by FACS or Cellomics Array Scan analysis, the inhibition of proliferation achieved by the compounds according to the invention is brought about primarily by preventing entry into the DNA synthesis phase. The treated cells arrest in the G1 phase of the cell cycle. Accordingly, the compounds according to the invention are also tested on other tumour cells. For example these compounds are active on the colon carcinoma cell line Colo205 and the breast cancer cell line DU4475 and can be used for these indications. This demonstrates the usefulness of the compounds according to the invention for treating various types of tumours.

Because of their biological properties the compounds of general formula (1) according to the invention, the tautomers, racemates, enantiomers, diastereomers, mixtures, polymorphs and the salts of all the above-mentioned forms are suitable for the treatment of diseases characterised by excessive or abnormal cell proliferation.

Diseases with excessive or abnormal cell proliferation are for example: viral infections (e.g. HIV and Kaposi's sarcoma); inflammatory and autoimmune diseases (e.g. Colitis, arthritis, Alzheimer's disease, glomerulonephritis and wound healing); bacterial, fungal and/or parasitic infections; leukaemias, lymphomas and solid tumours (e.g. Carcinomas and sarcomas), skin diseases (e.g. Psoriasis); diseases based on hyperplasia which are characterised by an increase in the number of cells (e.g. fibroblasts, hepatocytes, bones and bone marrow cells, cartilage or smooth muscle cells or epithelial cells (e.g. Endometrial hyperplasia)); bone diseases and cardiovascular diseases (e.g. restenosis and hypertrophy). They are also useful for protecting proliferating cells (e.g. hair, intestinal, blood and progenitor cells) from DNA damage caused by radiation, UV treatment and/or cytostatic treatment.

For example, the following cancers may be treated with compounds according to the invention, without being restricted thereto: brain tumours such as for example acoustic neurinoma, astrocytomas such as pilocytic astrocytomas, fibrillary astrocytoma, protoplasmic astrocytoma, gemistocytary astrocytoma, anaplastic astrocytoma and glioblastoma, brain lymphomas, brain metastases, hypophyseal tumour such as prolactinoma, HGH (human growth hormone) producing tumour and ACTH producing tumour (adrenocorticotropic hormone), craniopharyngiomas, medulloblastomas, meningeomas and oligodendrogliomas; nerve tumours (neoplasms) such as for example tumours of the vegetative nervous system such as neuroblastoma sympathicum, ganglioneuroma, paraganglioma (pheochromocytoma, chromaffinoma) and glomus-caroticum tumour, tumours on the peripheral nervous system such as amputation neuroma, neurofibroma, neurinoma (neurilemmoma, Schwannoma) and malignant Schwannoma, as well as tumours of the central nervous system such as brain and bone marrow tumours; intestinal cancer such as for example carcinoma of the rectum, colon, anus, small intestine and duodenum; eyelid tumours such as basalioma or basal cell carcinoma; pancreatic cancer or carcinoma of the pancreas; bladder cancer or carcinoma of the bladder; lung cancer (bronchial carcinoma) such as for example small-cell bronchial carcinomas (oat cell carcinomas) and non-small cell bronchial carcinomas such as plate epithelial carcinomas, adenocarcinomas and large-cell bronchial carcinomas; breast cancer such as for example mammary carcinoma such as infiltrating ductal carcinoma, colloid carcinoma, lobular invasive carcinoma, tubular carcinoma, adenocystic carcinoma and papillary carcinoma; non-Hodgkin's lymphomas (NHL) such as for example Burkitt's lymphoma, low-malignancy non-Hodgkin's lymphomas (NHL) and mucosis fungoides; uterine cancer or endometrial carcinoma or corpus carcinoma; CUP syndrome (Cancer of Unknown Primary); ovarian cancer or ovarian carcinoma such as mucinous, endometrial or serous cancer; gall bladder cancer; bile duct cancer such as for example Klatskin tumour; testicular cancer such as for example seminomas and non-seminomas; lymphoma (lymphosarcoma) such as for example malignant lymphoma, Hodgkin's disease, non-Hodgkin's lymphomas (NHL) such as chronic lymphatic leukaemia, leukaemic reticuloendotheliosis, immunocytoma, plasmocytoma (multiple myeloma), immunoblastoma, Burkitt's lymphoma, T-zone mycosis fungoides, large-cell anaplastic lymphoblastoma and lymphoblastoma; laryngeal cancer such as for example tumours of the vocal cords, supra-glottal, glottal and subglottal laryngeal tumours; bone cancer such as for example osteochondroma, chondroma, chondroblastoma, chondromyxoid fibroma, osteoma, osteoid osteoma, osteoblastoma, eosinophilic granuloma, giant cell tumour, chondrosarcoma, osteosarcoma, Ewing's sarcoma, reticulo-sarcoma, plasmocytoma, fibrous dysplasia, juvenile bone cysts and aneurysmatic bone cysts; head and neck tumours such as for example tumours of the lips, tongue, floor of the mouth, oral cavity, gums, palate, salivary glands, throat, nasal cavity, paranasal sinuses, larynx and middle ear; liver cancer such as for example liver cell carcinoma or hepatocellular carcinoma (HCC); leukaemias, such as for example acute leukaemias such as acute lymphatic/lymphoblastic leukaemia (ALL), acute myeloid leukaemia (AML); chronic leukaemias such as chronic lymphatic leukaemia (CLL), chronic myeloid leukaemia (CML); stomach cancer or gastric carcinoma such as for example papillary, tubular and mucinous adenocarcinoma, signet ring cell carcinoma, adenosquamous carcinoma, small-cell carcinoma and undifferentiated carcinoma; melanomas such as for example superficially spreading, nodular, lentigo-maligna and acral-lentiginous melanoma; renal cancer such as for example kidney cell carcinoma or hypernephroma or Grawitz's tumour; oesophageal cancer or carcinoma of the oesophagus; penile cancer; prostate cancer; throat cancer or carcinomas of the pharynx such as for example nasopharynx carcinomas, oropharynx carcinomas and hypopharynx carcinomas; retinoblastoma; vaginal cancer or vaginal carcinoma; plate epithelial carcinomas, adenocarcinomas, in situ carcinomas, malignant melanomas and sarcomas; thyroid carcinomas such as for example papillary, follicular and medullary thyroid carcinoma, as well as anaplastic carcinomas; spinalioma, epidormoid carcinoma and plate epithelial carcinoma of the skin; thymomas, cancer of the urethra and cancer of the vulva.

The new compounds may be used for the prevention, short-term or long-term treatment of the above-mentioned diseases, optionally also in combination with radiotherapy or other “state-of-the-art” compounds, such as e.g. cytostatic or cytotoxic substances, cell proliferation inhibitors, anti-angiogenic substances, steroids or antibodies.

The compounds of general formula (1) may be used on their own or in combination with other active substances according to the invention, optionally also in combination with other pharmacologically active substances.

Chemotherapeutic agents which may be administered in combination with the compounds according to the invention include, without being restricted thereto, hormones, hormone analogues and antihormones (e.g. tamoxifen, toremifene, raloxifene, fulvestrant, megestrol acetate, flutamide, nilutamide, bicalutamide, aminoglutethimide, cyproterone acetate, finasteride, buserelin acetate, fludrocortisone, fluoxymesterone, medroxyprogesterone, octreotide), aromatase inhibitors (e.g. anastrozole, letrozole, liarozole, vorozole, exemestane, atamestane), LHRH agonists and antagonists (e.g. goserelin acetate, luprolide), inhibitors of growth factors (growth factors such as for example “platelet derived growth factor” and “hepatocyte growth factor”, inhibitors are for example “growth factor” antibodies, “growth factor receptor” antibodies and tyrosinekinase inhibitors, such as for example gefitinib, imatinib, lapatinib and trastuzumab); antimetabolites (e.g. antifolates such as methotrexate, raltitrexed, pyrimidine analogues such as 5-fluorouracil, capecitabin and gemcitabin, purine and adenosine analogues such as mercaptopurine, thioguanine, cladribine and pentostatin, cytarabine, fludarabine); antitumour antibiotics (e.g. anthracyclins such as doxorubicin, daunorubicin, epirubicin and idarubicin, mitomycin-C, bleomycin, dactinomycin, plicamycin, streptozocin); platinum derivatives (e.g. cisplatin, oxaliplatin, carboplatin); alkylation agents (e.g. estramustin, meclorethamine, melphalan, chlorambucil, busulphan, dacarbazin, cyclophosphamide, ifosfamide, temozolomide, nitrosoureas such as for example carmustin and lomustin, thiotepa); antimitotic agents (e.g. Vinca alkaloids such as for example vinblastine, vindesin, vinorelbin and vincristine; and taxanes such as paclitaxel, docetaxel); topoisomerase inhibitors (e.g. epipodophyllotoxins such as for example etoposide and etopophos, teniposide, amsacrin, topotecan, irinotecan, mitoxantron) and various chemotherapeutic agents such as amifostin, anagrelid, clodronat, filgrastin, interferon alpha, leucovorin, rituximab, procarbazine, levamisole, mesna, mitotane, pamidronate and porfimer.

Suitable preparations include for example tablets, capsules, suppositories, solutions,—particularly solutions for injection (s.c., i.v., i.m.) and infusion—elixirs, emulsions or dispersible powders. The content of the pharmaceutically active compound(s) should be in the range from 0.1 to 90 wt.-%, preferably 0.5 to 50 wt.-% of the composition as a whole, i.e. in amounts which are sufficient to achieve the dosage range specified below. The doses specified may, if necessary, be given several times a day.

Suitable tablets may be obtained, for example, by mixing the active substance(s) with known excipients, for example inert diluents such as calcium carbonate, calcium phosphate or lactose, disintegrants such as corn starch or alginic acid, binders such as starch or gelatine, lubricants such as magnesium stearate or talc and/or agents for delaying release, such as carboxymethyl cellulose, cellulose acetate phthalate, or polyvinyl acetate. The tablets may also comprise several layers.

Coated tablets may be prepared accordingly by coating cores produced analogously to the tablets with substances normally used for tablet coatings, for example collidone or shellac, gum arabic, talc, titanium dioxide or sugar. To achieve delayed release or prevent incompatibilities the core may also consist of a number of layers. Similarly the tablet coating may consist of a number of layers to achieve delayed release, possibly using the excipients mentioned above for the tablets.

Syrups or elixirs containing the active substances or combinations thereof according to the invention may additionally contain a sweetener such as saccharine, cyclamate, glycerol or sugar and a flavour enhancer, e.g. a flavouring such as vanillin or orange extract. They may also contain suspension adjuvants or thickeners such as sodium carboxymethyl cellulose, wetting agents such as, for example, condensation products of fatty alcohols with ethylene oxide, or preservatives such as p-hydroxybenzoates.

Solutions for injection and infusion are prepared in the usual way, e.g. with the addition of isotonic agents, preservatives such as p-hydroxybenzoates, or stabilisers such as alkali metal salts of ethylenediamine tetraacetic acid, optionally using emulsifiers and/or dispersants, whilst if water is used as the diluent, for example, organic solvents may optionally be used as solvating agents or dissolving aids, and transferred into injection vials or ampoules or infusion bottles.

Capsules containing one or more active substances or combinations of active substances may for example be prepared by mixing the active substances with inert carriers such as lactose or sorbitol and packing them into gelatine capsules.

Suitable suppositories may be made for example by mixing with carriers provided for this purpose, such as neutral fats or polyethyleneglycol or the derivatives thereof.

Excipients which may be used include, for example, water, pharmaceutically acceptable organic solvents such as paraffins (e.g. petroleum fractions), vegetable oils (e.g. groundnut or sesame oil), mono- or polyfunctional alcohols (e.g. ethanol or glycerol), carriers such as e.g. natural mineral powders (e.g. kaolins, clays, talc, chalk), synthetic mineral powders (e.g. highly dispersed silicic acid and silicates), sugars (e.g. cane sugar, lactose and glucose) emulsifiers (e.g. lignin, spent sulphite liquors, methylcellulose, starch and polyvinylpyrrolidone) and lubricants (e.g. magnesium stearate, talc, stearic acid and sodium lauryl sulphate).

The preparations are administered by the usual methods, preferably by oral or transdermal route, most preferably by oral route. For oral administration the tablets may, of course contain, apart from the abovementioned carriers, additives such as sodium citrate, calcium carbonate and dicalcium phosphate together with various additives such as starch, preferably potato starch, gelatine and the like. Moreover, lubricants such as magnesium stearate, sodium lauryl sulphate and talc may be used at the same time for the tabletting process. In the case of aqueous suspensions the active substances may be combined with various flavour enhancers or colourings in addition to the excipients mentioned above.

For parenteral use, solutions of the active substances with suitable liquid carriers may be used.

The dosage for intravenous use is from 1-1000 mg per hour, preferably between 5 and 500 mg per hour.

However, it may sometimes be necessary to depart from the amounts specified, depending on the body weight, the route of administration, the individual response to the drug, the nature of its formulation and the time or interval over which the drug is administered. Thus, in some cases it may be sufficient to use less than the minimum dose given above, whereas in other cases the upper limit may have to be exceeded. When administering large amounts it may be advisable to divide them up into a number of smaller doses spread over the day.

The formulation examples that follow illustrate the present invention without restricting its scope:

Examples of Pharmaceutical Formulations

| | | |
|---|---|---|
| A) | Tablets | per tablet |

| | |
|---|---|
| active substance according to formula (1) | 100 mg |
| lactose | 140 mg |
| corn starch | 240 mg |
| polyvinylpyrrolidone | 15 mg |
| magnesium stearate | 5 mg |
| | 500 mg |

The finely ground active substance, lactose and some of the corn starch are mixed together. The mixture is screened, then moistened with a solution of polyvinylpyrrolidone in water, kneaded, wet-granulated and dried. The granules, the remaining corn starch and the magnesium stearate are screened and mixed together. The mixture is compressed to produce tablets of suitable shape and size.

| | | |
|---|---|---|
| B) | Tablets | per tablet |

| | |
|---|---|
| active substance according to formula (1) | 80 mg |
| lactose | 55 mg |
| corn starch | 190 mg |
| microcrystalline cellulose | 35 mg |
| polyvinylpyrrolidone | 15 mg |
| sodium-carboxymethyl starch | 23 mg |
| magnesium stearate | 2 mg |
| | 400 mg |

The finely ground active substance, some of the corn starch, lactose, microcrystalline cellulose and polyvinylpyrrolidone are mixed together, the mixture is screened and worked with the remaining corn starch and water to form a granulate which is dried and screened. The sodiumcarboxymethyl starch and the magnesium stearate are added and mixed in and the mixture is compressed to form tablets of a suitable size.

| | |
|---|---|
| C) | Ampoule solution |

| | | |
|---|---|---|
| active substance according to formula | 50 | mg |
| sodium chloride | 50 | mg |
| water for inj. | 5 | ml |

The active substance is dissolved in water at its own pH or optionally at pH 5.5 to 6.5 and sodium chloride is added to make it isotonic. The solution obtained is filtered free from pyrogens and the filtrate is transferred under aseptic conditions into ampoules which are then sterilised and sealed by fusion. The ampoules contain 5 mg, 25 mg and 50 mg of active substance.

1. A compound of the formula (1) 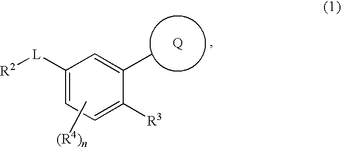
wherein
Q has a partial structure selected from the partial structures (i)-(iv) 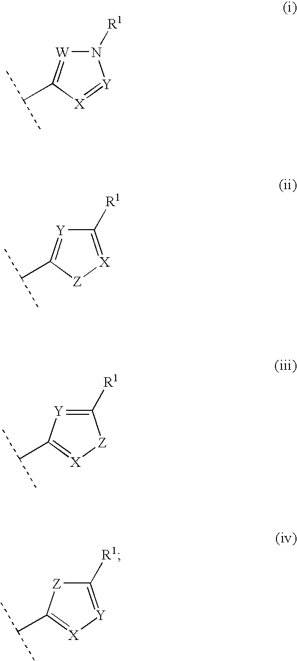
W, X and Y are each independently of one another selected from among ═CR^{5a}— and ═N—;
Z is independently selected in each case from among —NR⁶—, —O— and —S—;
L is selected from among —C(O)NH—, —NHC(O)—, —S(O)NH—, —S(O)₂NH—, —C(NH)NH—, —NHC(NH)—, —NHS(O)— and —NHS(O)₂—;
R¹ denotes a group optionally substituted by one or more identical or different R^{b} and/or R^{c}, selected from among C₃₋₁₀cycloalkyl, C₄₋₁₆cycloalkylalkyl, C₇₋₁₆arylalkyl, 5-12 membered heteroaryl, 6-18 membered heteroarylalkyl and 3-14 membered heterocycloalkyl;
R² is selected from among C₆₋₁₀aryl and 5-12 membered heteroaryl, the above-mentioned groups substituted by one or more identical or different R^{5b}, wherein at least one R^{5b} must be different from hydrogen;
R³ and each R⁴ are each independently selected from among hydrogen, halogen, —CN, —NO₂, —NR^{h}R^{h}, —OR^{h}, —C(O)R^{h}, —C(O)NR^{h}R^{h}, —SR^{h}, —S(O)R^{h}, —S(O)₂R^{h}, C₁₋₄alkyl, C₁₋₄haloalkyl, C₃₋₇cycloalkyl and 3-7 membered heterocycloalkyl;
each R^{5a} and R^{5b} is independently selected from among R^{a} and R^{b};
R⁶ is defined in the same way as R^{a};
n has the value 0, 1, 2 or 3;
each R^{a} independently denotes hydrogen or a group optionally substituted by one or more identical or different R^{b} and/or R^{c}, selected from among C₁₋₆alkyl, 2-6 membered heteroalkyl, C₁₋₆haloalkyl, C₃₋₁₀cycloalkyl, C₄₋₁₆cycloalkylalkyl, C₆₋₁₀aryl, C₇₋₁₆arylalkyl, 5-12 membered hetero-aryl, 6-18 membered heteroarylalkyl, 3-14 membered heterocycloalkyl and 4-14 membered heterocycloalkylalkyl;
each R^{b} denotes a suitable substituent and is independently selected from among —OR^{c}, —SR^{c}, —NR^{c}R^{c}, —ONR^{c}R^{c}, —N(OR^{c})R^{c}, —NR^{g}NR^{c}R^{c}, halogen, —CN, —NC, —OCN, —SCN, —NO, —NO₂, —N₃, —C(O)R^{c}, —C(O)OR^{c}, —C(O)NR^{c}R^{c}, —C(O)SR^{c}, —C(O)NR^{g}NR^{c}R^{c}, —C(O)NR^{g}OR^{c}, —[C(O)]₂NR^{c}R^{c}, —[C(O)NR^{g}]₂R^{c}, —C(S)R^{c}, —C(S)OR^{c}, —C(S)NR^{c}R^{c}, —C(S)SR^{c}, —C(NR^{g})R^{c}, —N═CR^{c}R^{c}, —C(NR^{g})OR^{c}, —C(NR^{g})NR^{c}R^{c}, —C(NR^{g})SR^{c}, —C(NR^{g})NR^{g}NR^{c}R^{c}, —C(NOR^{g})R^{c}, —C(NOR^{g})NR^{c}R^{c}, —C(NNR^{g}R^{g})R^{c}, —C[NNR^{g}C(O)NR^{g}R^{g}]R^{c}, —OS(O)R^{c}, —OS(O)OR^{c}, —OS(O)NR^{c}R^{c}, —OS(O)₂R^{c}, —OS(O)₂OR^{c}, —OS(O)₂NR^{c}R^{c}, —OC(O)R^{c}, —OC(O)OR^{c}, —OC(O)SR^{c}, —OC(O)NR^{c}R^{c}, —O[C(O)]₂NR^{c}R^{c}, —O[C(O)NR^{g}]₂NR^{c}R^{c}, —OC(S)R^{c}, —OC(NR^{g})R^{c}, —OC(NR^{g})NR^{c}R^{c}, —ONR^{g}C(O)R^{c}, —S(O)R^{c}, —S(O)OR^{c}, —S(O)NR^{c}R^{c}, —S(O)₂R^{c}, —S(O)₂OR^{c}, —S(O)₂NR^{c}R^{c}, —[S(O)₂]₂NR^{c}R^{c}, —SC(O)R^{c}, —SC(O)OR^{c}, —SC(O)NR^{c}R^{c}, —SC(S)R^{c}, —SC(NR^{g})R^{c}, —SC(NR^{g})NR^{c}R^{c}, —NR^{g}C(O)R^{c}, —NR^{g}C(O)OR^{c}, —NR^{g}C(O)NR^{c}R^{c}, —NR^{g}C(O)SR^{c}, —NR^{g}C(O)NR^{g}NR^{c}R^{c}, —NR^{g}C(S)R^{c}, —NR^{g}C(S)NR^{c}R^{c}, —NR^{g}C(NR^{g})R^{c}, —N═CR^{c}NR^{c}R^{c}, —NR^{g}C(NR^{g})OR^{c}, —NR^{g}C(NR^{g})NR^{c}R^{c}, —NR^{g}C(NR^{g})SR^{c}, —NR^{g}C(NOR^{g})R^{c}, —NR^{g}S(O)R^{c}, —NR^{g}S(O)OR^{c}, —NR^{g}S(O)₂R^{c}, —NR^{g}S(O)₂OR^{c}, —NR^{g}S(O)₂NR^{c}R^{c}, —NR^{g}NR^{g}C(O)R^{c}, —NR^{g}NR^{g}C(O)NR^{c}R^{c}, —NR^{g}NR^{g}C(NR^{g})R^{c}, —NR^{g}[C(O)]₂R^{c}, —NR^{g}[C(O)]₂OR^{c}, —NR^{g}[C(O)]₂NR^{c}R^{c}, —[NR^{g}C(O)]₂R^{c}, —[NR^{g}C(O)]₂OR^{c}, —NR^{g}[S(O)₂]₂R^{c}, —N(OR^{g})C(O)R^{c}, —N[C(O)R^{c}]NR^{c}R^{c}, —N[C(O)R^{c}]₂, —N[S(O)₂R^{c}]₂, —N{[C(O)]₂R^{c}}₂, —N{[C(O)]₂OR^{c}}₂ and —N{[C(O)]₂NR^{c}R^{c}}₂ and the bivalent substituents ═O, ═S, ═NR^{g}, ═NOR^{g}, ═NNR^{g}R^{g} and ═NNR^{g}C(O)NR^{g}R^{g}, while these bivalent substituents may only be substituents in non-aromatic ring systems;
each R^{c} independently denotes hydrogen or a group optionally substituted by one or more identical or different R^{d} and/or R^{c}, selected from among C₁₋₆alkyl, 2-6 membered heteroalkyl, C₁₋₆haloalkyl, C₃₋₁₀cycloalkyl, C₄₋₁₆cycloalkylalkyl, C₆₋₁₀aryl, C₇₋₁₆arylalkyl, 5-12 membered hetero-aryl, 6-18 membered heteroarylalkyl, 3-14 membered heterocycloalkyl and 4-14 membered heterocycloalkylalkyl;
each R^{d} denotes a suitable substituent and is independently selected from among —OR^{e}, —SR^{e}, —NR^{e}R^{e}, —ONR^{e}R^{e}, —N(OR^{e})R^{e}, —N(R^{g})NR^{e}R^{e}, halogen, —CN, —NC, —OCN, —SCN, —NO, —NO₂, —N₃, —C(O)R^{e}, —C(O)OR^{e}, —C(O)NR^{e}R^{e}, —C(O)SR^{e}, —C(O)NR^{g}NR^{e}R^{e}, —C(O)NR^{g}OR^{e}, —[C(O)]₂NR^{e}R^{e}, —[C(O)NR^{g}]₂R^{e}, —C(S)R^{e}, —C(S)OR^{e}, —C(S)NR^{e}R^{e}, —C(S)SR^{e}, —C(NR^{g})R^{e}, —N═CR^{e}R^{e}, —C(NR^{g})OR^{e}, —C(NR^{g})NR^{e}R^{e}, —C(NR^{g})SR^{e}, —C(NR^{g})NR^{g}NR^{e}R^{e}, —C(NOR^{g})R^{e}, —C(NOR^{g})NR^{e}R^{e}, —C(NNR^{g}R^{g})R^{e}, —C[NNR^{g}C(O)NR^{g}R^{g}]R^{e}, —OS(O)R^{e}, —OS(O)OR^{e}, —OS(O)NR^{e}R^{e}, —OS(O)₂R^{e}, —OS(O)₂OR^{e}, —OS(O)₂NR^{e}R^{e}, —OC(O)R^{e}, —OC(O)OR^{e}, —OC(O)SR^{e}, —OC(O)NR^{e}R^{e}, —O[C(O)]₂NR^{e}R^{e}, —O[C(O)NR^{g}]₂NR^{e}R^{e}, —OC(S)R^{e}, —OC(NR^{g})R^{e}, —OC(NR^{g})NR^{e}R^{e}, —ONR^{g}C(O)R^{e}, —S(O)R^{e}, —S(O)OR^{e}, —S(O)NR^{e}R^{e}, —S(O)₂R^{e}, —S(O)₂OR^{e}, —S(O)₂NR^{e}R^{e}, —[S(O)₂]₂NR^{e}R^{e}, —SC(O)R^{e}, —SC(O)OR^{e}, —SC(O)NR^{e}R^{e}, —SC(S)R^{e}, —SC(NR^{g})R^{e}, —SC(NR^{g})NR^{e}R^{e}, —NR^{g}C(O)R^{e}, —NR^{g}C(O)OR^{e}, —NR^{g}C(O)NR^{e}R^{e}, —NR^{g}C(O)SR^{e}, —NR^{g}C(O)NR^{g}NR^{e}R^{e}, —NR^{g}C(S)R^{e}, —NR^{g}C(S)NR^{e}R^{e}, —NR^{g}C(NR^{g})R^{e}, —N═CR^{e}NR^{e}R^{e}, —NR^{g}C(NR^{g})OR^{e}, —NR^{g}C(NR^{g})NR^{e}R^{e}, —NR^{g}C(NR^{g})SR^{e}, —NR^{g}C(NOR^{g})R^{e}, —NR^{g}S(O)R^{e}, —NR^{g}S(O)OR^{e}, —NR^{g}S(O)₂R^{e}, —NR^{g}S(O)₂OR^{e}, —NR^{g}S(O)₂NR^{e}R^{e}, —NR^{g}NR^{g}C(O)R^{e}, —NR^{g}NR^{g}C(O)NR^{e}R^{e}, —NR^{g}NR^{g}C(NR^{g})R^{e}, —NR^{g}[C(O)]₂R^{e}, —NR^{g}[C(O)]₂OR^{e}, —NR^{g}[C(O)]₂NR^{e}R^{e}, —[NR^{g}C(O)]₂R^{e}, —[NR^{g}C(O)]₂OR^{e}, —NR^{g}[S(O)₂]₂R^{e}, —N(OR^{g})C(O)R^{e}, —N[C(O)R^{e}]NR^{e}R^{e}, —N[C(O)R^{e}]₂, —N[S(O)₂R^{e]}₂, —N{[C(O)]₂R^{e}}₂, —N{[C(O)]₂OR^{e}}₂ and —N{[C(O)]₂NR^{e}R^{e}}₂ and the bivalent substituents ═O, ═S, ═NR^{g}, ═NOR^{g}, ═NNR^{g}R^{g} and ═NNR^{g}C(O)NR^{g}R^{g}, while these bivalent substituents may only be substituents in non-aromatic ring systems;
each R^{e} independently denotes hydrogen or a group optionally substituted by one or more identical or different R^{f} and/or R^{g}, selected from among C₁₋₆alkyl, 2-6 membered heteroalkyl, C₁₋₆haloalkyl, C₃₋₁₀cycloalkyl, C₄₋₁₆cycloalkylalkyl, C₆₋₁₀aryl, C₇₋₁₆arylalkyl, 5-12 membered heteroaryl, 6-18 membered heteroarylalkyl, 3-14 membered heterocycloalkyl and 4-14 membered heterocycloalkylalkyl;
each R^{f} denotes a suitable substituent and is independently selected from among —OR^{g}, —SR^{g}, —NR^{g}R^{g}, —ONR^{g}R^{g}, —N(OR^{g})R^{g}, —N(R^{h})NR^{g}R^{g}, halogen, —CN, —NC, —OCN, —SCN, —NO, —NO₂, —N₃, —C(O)R^{g}, —C(O)OR^{g}, —C(O)NR^{g}R^{g}, —C(O)SR^{g}, —C(O)NR^{h}NR^{g}R^{g}, —C(O)NR^{h}OR^{g}, [C(O)]₂NR^{g}R^{g}, —[C(O)NR^{h}]₂R^{g}, —C(S)R^{g}, —C(S)OR^{g}, —C(S)NR^{g}R^{g}, —C(S)SR^{g}, —C(NR^{h})R^{g}, —N═CR^{g}R^{g}, —C(NR^{h})OR^{g}, —C(NR^{h})NR^{g}R^{g}, —C(NR^{h})SR^{g}, —C(NR^{h})NR^{h}NR^{g}R^{g}, —C(NOR^{h})R^{g}, —C(NOR^{h})NR^{g}R^{g}, —C(NNR^{h}R^{h})R^{g}, —C[NNR^{h}C(O)NR^{h}R^{h}]R^{g}, —OS(O)R^{g}, —OS(O)OR^{g}, —OS(O)NR^{g}R^{g}, —OS(O)₂R^{g}, —OS(O)₂OR^{g}, —OS(O)₂NR^{g}R^{g}, —OC(O)R^{g}, —OC(O)OR^{g}, —OC(O)SR^{g}, —OC(O)NR^{g}R^{g}, —O[C(O)]₂NR^{g}R^{g}, —O[C(O)NR^{h}]₂NR^{g}R^{g}, —OC(S)R^{g}, —OC(NR^{h})R^{g}, —OC(NR^{h})NR^{g}R^{g}, —ONR^{h}C(O)R^{g}, —S(O)R^{g}, —S(O)OR^{g}, —S(O)NR^{g}R^{g}, —S(O)₂R^{g}, —S(O)₂OR^{g}, —S(O)₂NR^{g}R^{g}, —[S(O)₂]₂NR^{g}R^{g}, —SC(O)R^{g}, —SC(O)OR^{g}, —SC(O)NR^{g}R^{g}, —SC(S)R^{g}, —SC(NR^{h})R^{g}, —SC(NR^{h})NR^{g}R^{g}, —NR^{h}C(O)R^{g}, —NR^{h}C(O)OR^{g}, —NR^{h}C(O)NR^{g}R^{g}, —NR^{h}C(O)SR^{g}, —NR^{h}C(O)NR^{h}NR^{g}R^{g}, —NR^{h}C(S)R^{g}, —NR^{h}C(S)NR^{g}R^{g}, —NR^{h}C(NR^{h})R^{g}, —N═CR^{g}NR^{g}R^{g}, —NR^{h}C(NR^{h})OR^{g}, —NR^{h}C(NR^{h})NR^{g}R^{g}, —NR^{h}C(NR^{h})SR^{g}, —NR^{h}C(NOR^{h})R^{g}, —NR^{h}S(O)R^{g}, —NR^{h}S(O)OR^{g}, —NR^{h}S(O)₂R^{g}, —NR^{h}S(O)₂OR^{g}, —NR^{h}S(O)₂NR^{g}R^{g}, —NR^{h}NR^{h}C(O)R^{g}, —NR^{h}NR^{h}C(O)NR^{g}R^{g}, —NR^{h}NR^{h}C(NR^{h})R^{g}, —NR^{h}[C(O)]₂R^{g}, —NR^{h}[C(O)]₂OR^{g}, —NR^{h}[C(O)]₂NR^{g}R^{g}, —[NR^{h}C(O)]₂R^{g}, [NR^{h}C(O)]₂OR^{g}, —NR^{h}[S(O)₂]₂R^{g}, —N(OR^{h})C(O)R^{g}, —N[C(O)R^{g}]NR^{g}R^{g}, —N[C(O)R^{g}]₂, —N[S(O)₂R^{g}]₂, —N{[C(O)]₂R^{g}}₂, —N{[C(O)]₂OR^{g}}₂ and —N{[C(O)]₂NR^{g}R^{g}}₂ and the bivalent substituents ═O, ═S, ═NR^{h}, ═NOR^{h}, ═NNR^{h}R^{h} and ═NNR^{h}C(O)NR^{h}R^{h}, while these bivalent substituents may only be substituents in non-aromatic ring systems;
each R^{g} independently denotes hydrogen or a group optionally substituted by one or more identical or different R^{h}, selected from among C₁₋₆alkyl, 2-6 membered heteroalkyl, C₁₋₆haloalkyl, C₃₋₁₀cycloalkyl, C₄₋₁₆cycloalkylalkyl, C₆₋₁₀aryl, C₇₋₁₆arylalkyl, 5-12 membered heteroaryl, 6-18 membered heteroarylalkyl, 3-14 membered heterocycloalkyl and 4-14 membered heterocycloalkylalkyl;
each R^{h} is independently selected from among hydrogen, C₁₋₆alkyl, 2-6 membered heteroalkyl, C₁₋₆haloalkyl, C₃₋₁₀cycloalkyl, C₄₋₁₆cycloalkylalkyl, C₆₋₁₀aryl, C₇₋₁₆arylalkyl, 5-12 membered heteroaryl, 6-18 membered heteroarylalkyl, 3-14 membered heterocycloalkyl and 4-14 membered heterocycloalkylalkyl;
with the proviso that
N-[2-amino-5-(5-isoxazol-3-yl-thiophen-2-yl)-phenyl]-4-methoxy-benzamide and
N-{3-[2-(5-carbamimidoyl-2-methylsulphanyl-thiophen-3-yl)-thiazol-4-yl]-phenyl}-4-fluoro-benzamide are excluded,
while the compounds (1) may optionally also be present in the form of the tautomers, the racemates, the enantiomers, the diastereomers, the mixtures thereof or as pharmacologically acceptable salts of all the above-mentioned forms.

2. The compound according to claim 1, wherein
L is selected from among —C(O)NH— and —NHC(O)—.

3. The compound according to claim 1, wherein
neither R³ nor R⁴ denotes —OH or —NH₂.

4. The compound according to claim 3, wherein
n has the value 0.

5. The compound according to claim 4, wherein
the heteroaromatic five-membered ring in the partial structures (i)-(iv) is not pyrazole.

6. The compound according to claim 4, wherein
the heteroaromatic five-membered ring in the partial structures (i)-(iv) is not imidazole.

7. The compound according to claim 4, wherein
the heteroaromatic five-membered ring in the partial structures (ii)-(iv) is not thiophene.

8. The compound according to claim 4, wherein
the heteroaromatic five-membered ring in the partial structures (i)-(iv) has at least two heteroatoms.

9. The compound according to claim 4, wherein
Q has a partial structure selected from the partial structures (v)-(xiii) 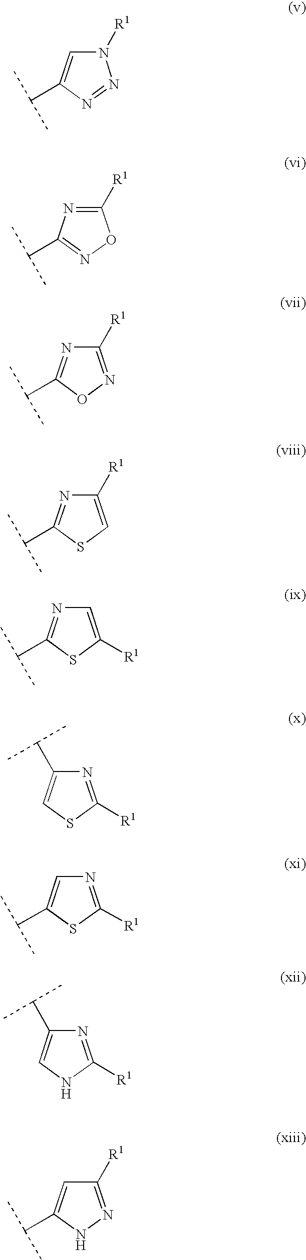
and
R¹ is defined as in claim 1.

10. The compound according to claim 9, wherein
Q has the partial structure (v) 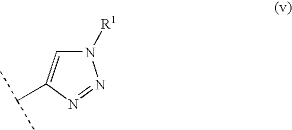
and
R¹ is defined as in claim 1.

11. The compound according to claim 10, wherein
R¹ denotes a group optionally substituted by one or more identical or different R^{b1} and/or R^{c1}, selected from among C₃₋₁₀cycloalkyl, C₄₋₁₆cycloalkylalkyl, C₇₋₁₆arylalkyl, 5-12 membered heteroaryl, 6-18 membered heteroarylalkyl and 3-14 membered heterocycloalkyl;
each R^{b1} denotes a suitable substituent and is independently selected from among —OR^{c1}, —SR^{c1}, —NR^{c1}R^{c1} halogen, —CN, —NO₂, —C(O)R^{c1}, —C(O)OR^{c1}, —C(O)NR^{c1}R^{c1}, —NHC(O)R^{c1}, —NHC(O)OR^{c1}, —NHC(O)NR^{c1}R^{c1}, S(O)R^{c1}, S(O)₂R^{c1} as well as the bivalent substituent ═O, while this bivalent substituent may only be a substituent in non-aromatic ring systems;
each R^{c1} independently denotes hydrogen or a group optionally substituted by one or more identical or different R^{d1} and/or R^{e1}, selected from among C₁₋₆alkyl, 2-6 membered heteroalkyl, C₁₋₆haloalkyl, C₃₋₁₀cycloalkyl, C₄₋₁₆cycloalkylalkyl, C₆₋₁₀aryl, C₇₋₁₆arylalkyl, 5-12 membered heteroaryl, 6-18 membered heteroarylalkyl, 3-14 membered heterocycloalkyl and 4-14 membered heterocycloalkylalkyl;
each R^{d1} denotes a suitable substituent and is independently selected from among —OR^{e1}, —NR^{e1}R^{e1}, halogen, —CN, —NO₂, —C(O)R^{e1}, —C(O)OR^{e1}, —C(O)NR^{e1}R^{e1}, —OC(O)R^{e1}, —OC(O)OR^{e1}, —OC(O)NR^{e1}R^{e1}, —NHC(O)R^{e1}, —NHC(O)OR^{e1}, —NHC(O)NR^{e1}R^{e1} as well as the bivalent substituent ═O, while this bivalent substituent may only be a substituent in non-aromatic ring systems;
each R^{e1} is independently selected from among hydrogen, C₁₋₆alkyl, 2-6 membered heteroalkyl, C₁₋₆haloalkyl, C₃₋₁₀cycloalkyl, C₄₋₁₆cycloalkylalkyl, C₆₋₁₀aryl, C₇₋₁₆arylalkyl, 5-12 membered heteroaryl, 6-18 membered heteroarylalkyl, 3-14 membered heterocycloalkyl and 4-14 membered heterocycloalkylalkyl.

12. The compound according to claim 11, wherein
R¹ is a group optionally substituted by one or more identical or different R^{b1} and/or R^{c1}, selected from among 5-12 membered heteroaryl and 3-14 membered heterocycloalkyl.

13. The compound according to claim 10, wherein
R¹ is a group optionally substituted by one or more identical or different R^{b} and/or R^{c}, selected from among 5-12 membered heteroaryl and 3-14 membered heterocycloalkyl, and
R^{b} and R^{c} are defined as in claim 1.

14. The compound according to claim 12 or 13, wherein
R¹ is selected from among pyridyl, pyrimidyl, thiazolyl, imidazolyl, triazolyl, pyrazolyl, pyrrolyl, furanyl, phenyl, benzyl, imidazo[2.1-b]thiazolyl and imidazo[1,2-a]pyridyl and all the above-mentioned ring systems are optionally mono- or polysubstituted.

15. The compound according to claim 14, wherein
R² is selected from among phenyl, pyridyl, pyrazolyl, isoxazolyl, thiazolyl, imidazolyl and oxazolyl, all the above-mentioned groups are optionally substituted by one or more identical or different R^{5b} and
R^{5b} is defined as in claim 1.

16. The compound according to claim 15, wherein
each R^{5b} is independently selected from among R^{a2} and R^{b2};
each R^{a2} is a group optionally substituted by one or more identical or different R^{b2} and/or R^{c2}, selected from among C₁₋₆alkyl, 2-6 membered heteroalkyl, C₁₋₆haloalkyl, C₃₋₁₀cycloalkyl, C₄₋₁₆cycloalkylalkyl, C₆₋₁₀aryl, C₇₋₁₆arylalkyl, 5-12 membered heteroaryl, 6-18 membered heteroarylalkyl, 3-14 membered heterocycloalkyl and 4-14 membered heterocycloalkylalkyl;
each R^{b2} denotes a suitable substituent and is independently selected from among —OR^{c2}, —SR^{c2}, —NR^{c2}R^{c2}, halogen, —CF₃, —CN, —NO₂, —S(O)R^{c2}, —S(O)₂R^{c2}, —S(O)NR^{c2}R^{c2}, —S(O)₂NR^{c2}R^{c2}, —C(O)R^{c2}, —C(O)OR^{c2}, —C(O)NR^{c2}R^{c2}, —OC(O)R^{c2}, —OC(O)OR^{c2}, —OC(O)NR^{c2}R^{c2}, —NHC(O)R^{c2}, —NHS(O)₂R^{c2}, —NHC(O)OR^{c2}, —NHC(O)NR^{c2}R^{c2}, N═CR^{c2}R^{c2}, —N═CR^{c2}NR^{c2}R^{c2} as well as the bivalent substituent ═O, wherein this bivalent substituent may only be a substituent in non-aromatic ring systems;
each R^{c2} independently denotes hydrogen or a group optionally substituted by one or more identical or different R^{d2} and/or R^{e2}, selected from among C₁₋₆alkyl, 2-6 membered heteroalkyl, C₁₋₆haloalkyl, C₃₋₁₀cycloalkyl, C₄₋₁₆cycloalkylalkyl, C₆₋₁₀aryl, C₇₋₁₆arylalkyl, 5-12 membered heteroaryl, 6-18 membered heteroarylalkyl, 3-14 membered heterocycloalkyl and 4-14 membered heterocycloalkylalkyl;
each R^{d2} denotes a suitable substituent and is independently selected from among —OR^{e2}, NR^{e2}R^{e2}, halogen, —CN, —NO₂, —C(O)R^{e2}, —C(O)OR^{e2}, —C(O)NR^{e2}R^{e2}, —OC(O)R^{e2}, —OC(O)OR^{e2}, —OC(O)NR^{e2}R^{e2}, —NHC(O)R^{e2}, —NHC(O)OR^{e2}, —NHC(O)NR^{e2}R^{e2} as well as the bivalent substituent ═O, wherein this bivalent substituent may only be a substituent in non-aromatic ring systems;
each R^{e2} is independently selected from among hydrogen, C₁₋₆alkyl, 2-6 membered heteroalkyl, C₁₋₆haloalkyl, C₃₋₁₀cycloalkyl, C₄₋₁₆cycloalkylalkyl, C₆₋₁₀aryl, C₇₋₁₆arylalkyl, 5-12 membered heteroaryl, 6-18 membered heteroarylalkyl, 3-14 membered heterocycloalkyl and 4-14 membered heterocycloalkylalkyl.

17-20. (canceled)

21. A method of treating cancer, infections, inflammations or autoimmune diseases comprising administering a therapeutically effective amount of a compound according to claim 1.

22. A pharmaceutical composition comprising a therapeutically effective amount of a compound of the formula (1) according to claim 1 and one or more pharmaceutically acceptable excipients and/or carriers.

23. The pharmaceutical composition according to claim 22 further comprising at least one cytostatic or cytotoxic active substance different from the formula (1) in claim 1.
